(19) United States
(12) Patent Application Publication
(10) Pub. No.: US 2016/0160213 A1
(43) Pub. Date: Jun. 9, 2016

(54) METHODS OF TREATING CANCER AND PREVENTING CANCER DRUG RESISTANCE

(71) Applicant: Genentech, Inc., South San Francisco, CA (US)

(72) Inventors: Marie Classon, Mill Valley, CA (US); Jean-Philippe Stephan, L'Etang-La-Ville (FR)

(73) Assignee: GENENTECH, INC., South San Francisco, CA (US)

(21) Appl. No.: 14/853,752

(22) Filed: Sep. 14, 2015

Related U.S. Application Data
(63) Continuation of application No. PCT/US14/28759, filed on Mar. 14, 2014.
(60) Provisional application No. 61/785,645, filed on Mar. 14, 2013.

(51) Int. Cl. C12N 15/113; A61K 31/437; A61K 31/5377; A61K 31/165; A61K 31/713; A61K 31/496; A61K 31/337; A61K 31/517

(52) U.S. Cl. CPC C12N 15/113; A61K 31/337; A61K 31/437; A61K 31/517; A61K 31/165; A61K 31/713; A61K 31/496; A61K 31/5377; C12N 2320/31; C12N 2310/14

(57) ABSTRACT

Provided herein are methods of treating and/or preventing cancer drug resistance using modulators of chromatin modifiers (e.g., antagonists of chromatin modifiers) described herein.

### CROSS REFERENCE TO RELATED APPLICATIONS

This application is a continuation of International Application No. PCT/US2014/028759 having an international filing date of Mar. 14, 2014, the entire contents of which are incorporated herein by reference, and which claims benefit under 35 U.S.C. §119 to U.S. Provisional Patent Application No. 61/785,645 filed Mar. 14, 2013, the entire contents of which are incorporated herein by reference.

### FIELD

Provided herein are methods of treating and/or preventing cancer drug resistance using modulators of chromatin modifiers (e.g., antagonists of chromatin modifiers) as described herein.

### BACKGROUND

The relatively rapid acquisition of resistance to cancer drugs remains a key obstacle to successful cancer therapy. Substantial efforts to elucidate the molecular basis for such drug resistance have revealed a variety of mechanisms, including drug efflux, acquisition of drug binding-deficient mutants of the target, engagement of alternative survival pathways, and epigenetic alterations. Such mechanisms are generally believed to reflect the existence of rare, stochastic, resistance-conferring genetic alterations within a tumor cell population that are selected during drug treatment. See Sharma et al., Cell 141(1):69-80 (2010). An increasingly observed phenomenon in cancer therapy is the so-called “re-treatment response.” For example, some non-small cell lung cancer (NSCLC) patients who respond well to treatment with EGFR (epidermal growth factor receptor) tyrosine kinase inhibitors (TKIs), and who later experience therapy failure, demonstrate a second response to EGFR TKI re-treatment after a “drug holiday.” See Kurata et al., Ann. Oncol. 15:173-174 (2004); Yano et al., Oncol. Res. 15:107-111 (2005). Similar re-treatment responses are well established for several other anti-cancer agents. See Cara and Tannock, Ann. Oncol. 12:23-27 (2001). Such findings suggest that acquired resistance to cancer drugs may involve a reversible “drug-tolerant” state, whose mechanistic basis remains to be established.

The existence of a reversibly “drug-tolerant” cell population within various human tumor cell lines has been shown to be maintained via engagement of IGF-1 receptor signaling and an altered chromatin state that requires the histone demethylase KDM5A. While some specific resistance-conferring mutations have indeed been identified in many cancer patients demonstrating acquired drug resistance, the relative contribution of mutational and non-mutational mechanisms to drug resistance, and the role of tumor cell subpopulations remain somewhat unclear. New treatment methods are needed to successfully address heterogeneity within cancer cell populations and the emergence of cancer cells resistant to drug treatments.

### SUMMARY

Provided herein are methods of using modulators of chromatin modifiers (e.g., antagonists of chromatin modifiers), for example, for treating cancer and/or preventing drug resistance in an individual. In some embodiments, the individual is selected for treatment with a cancer therapy agent (e.g., targeted therapies, chemotherapies, and/or radiotherapies). In some embodiments, the individual starts treatment comprising administration of the modulator of the chromatin modifier prior to treatment with the cancer therapy agent. In some embodiments, the individual concurrently receives treatment comprising the modulator of the chromatin modifier and the cancer therapy agent. In some embodiments, the chromatin modifier increases period of cancer sensitivity and/or delay development of cancer resistance.

In another aspect, provided herein are combination therapies using modulators of chromatin modifiers (e.g., antagonists of chromatin modifiers) and cancer therapy agents (e.g., targeted therapies, chemotherapies, and/or radiotherapies).

In particular, provided herein are methods of treating cancer in an individual comprising administering to the individual (a) a modulator of a chromatin modifier and (b) a cancer therapy agent. In some embodiments, the respective amounts of the modulator of the chromatin and the cancer therapy agent are effective to increase the period of cancer sensitivity and/or delay the development of cancer cell resistance to the cancer therapy agent. In some embodiments, the respective amounts of the modulator of the chromatin modifier and the cancer therapy agent are effective to increase efficacy of a cancer treatment comprising the cancer therapy agent. For example, in some embodiments, the respective amounts of the modulator of the chromatin modifier and the cancer therapy agent are effective to increased efficacy compared to a standard treatment comprising administering an effective amount of cancer therapy agent without (in the absence of) the modulator of the chromatin modifier. In some embodiments, the respective amounts of the modulator of the chromatin modifier and the cancer therapy agent are effective to increased response (e.g., complete response) compared to a standard treatment comprising administering an effective amount of cancer therapy without (in the absence of) the modulator of the chromatin modifier. In some embodiments, the modulator of the chromatin modifier is an antagonist of the chromatin modifier.

Also provided herein are methods of increasing efficacy of a cancer treatment comprising a cancer therapy agent in an individual comprising administering to the individual (a) an effective amount of a modulator of a chromatin modifier and (b) an effective amount of the cancer therapy agent.

Provided herein are methods of treating cancer in an individual wherein cancer treatment comprising administering to the individual (a) an effective amount of a modulator of a chromatin modifier and (b) an effective amount of a cancer therapy agent, wherein the cancer treatment has increased efficacy compared to a standard treatment comprising administering an effective amount of cancer therapy agent without (in the absence of) the chromatin modifier.

In addition, provided herein are methods of delaying and/or preventing development of cancer resistant to a cancer therapy agent in an individual, comprising administering to the individual (a) an effective amount of a modulator of a chromatin modifier and (b) an effective amount of the cancer therapy agent.

Provided herein are methods of treating an individual with cancer who has increased likelihood of developing resistance to a cancer therapy agent comprising administering to the individual (a) an effective amount of a modulator of a chromatin modifier and (b) an effective amount of the cancer therapy agent.

Further provided herein are methods of increasing sensitivity to a cancer therapy agent in an individual with cancer comprising administering to the individual (a) an effective amount of a modulator of a chromatin modifier and (b) an effective amount of the cancer therapy agent.

Provided herein are also methods extending the period of a cancer therapy agent sensitivity in an individual with cancer comprising administering to the individual (a) an effective amount of a modulator of a chromatin modifier and (b) an effective amount of the cancer therapy agent.

Provided herein are methods of extending the duration of response to a cancer therapy agent in an individual with cancer comprising administering to the individual (a) an effective amount of a modulator of a chromatin modifier and (b) an effective amount of the cancer therapy agent.

In some embodiments of any of the methods, the modulator of the chromatin modifier is an antagonist of a chromatin modifier.

In some embodiments of any of the methods, the chromatin modifier is a member of polycomb repressive complex (PRC). In some embodiments, the member of PRC is a member of polycomb repressive complex 1 (PRC1). In some embodiments, the member of PRC1 is one or more of RING1B, CBX3, CBX6, and CBX8. In some embodiments, the member of PRC is a member of polycomb repressive complex 2 (PRC2). In some embodiments, the member of PRC2 is EZH2, SUZ12, and/or EED. In some embodiments, the member of the PRC2 is EZH2. In some embodiments, the member of the PRC2 is SUZ12. In some embodiments, the member of the PRC2 is EED.

In some embodiments of any of the methods, the chromatin modifier is an EZH2 inhibitor. In some embodiments, the EZH2 inhibitor is a small molecule EZH2 inhibitor. In some embodiments, the small molecule EZH2 inhibitor is N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide or a pharmaceutically acceptable salt thereof. In some embodiments, the small molecule EZH2 inhibitor is (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide or a pharmaceutically acceptable salt thereof.

In some embodiments of any of the methods, the chromatin modifier is a member of nucleosome remodeling and deacetylation complex (NuRD). In some embodiments, the member of NuRD is one or more of CHD4, RBBP4, HDAC1, HDAC2, and HDAC3. In some embodiments, the member of NuRD is HDAC2 and/or HDAC3.

In some embodiments of any of the methods, the chromatin modifier is an ubiquitin-conjugating enzyme. In some embodiments, the ubiquitin-conjugating enzyme is UBE2A and/or UBE2B.

In some embodiments of any of the methods, the chromatin modifier is one or more of ATRX, MYST4, CDYL, LRWD1, CHD7, PHF10, PHF12, PHF23, CHD1, MGEA5, MLLT10, SIRT4, TP53BP1, BRDT, CBX6, EVI1, GTF3C4, HIRA, MPHOSPH8, NCOA1, RBBP5, TDRD7, and ZCWPW1. In some embodiments of any of the methods, the chromatin modifier is one or more of ATRX, MYST4, CDYL, LRWD1, CHD7, PHF10, PHF12, PHF23, and CHD1. In some embodiments of any of the methods, the chromatin modifier is one or more of MGEA5, MLLT10, SIRT4, TP53BP1, ATRX, BRDT, CBX6, CHD1, EVI1, GTF3C4, HIRA, MPHOSPH8, NCOA1, RBBP5, TDRD7, and ZCWPW1.

In some embodiments of any of the methods, the cancer therapy agent is a targeted therapy. In some embodiments, the targeted therapy is an EGFR antagonist. In some embodiments of any of the methods, the EGFR antagonist is N-(3-ethynylphenyl)-6,7-bis(2-methoxyethoxy)-4-quinazolinamine and/or a pharmaceutical acceptable salt thereof. In some embodiments, the EGFR antagonist is N-(3-ethynylphenyl)-6,7-bis(2-methoxyethoxy)-4-quinazolinamine.

In some embodiments of any of the methods, the cancer therapy agent is a chemotherapy. In some embodiments, the chemotherapy is a taxane. In some embodiments, the taxane is paclitaxel.

In some embodiments, the modulator of the chromatin modifier and the cancer therapy agent are administered concomitantly.

In some embodiments of any of the methods, the cancer is lung cancer. In some embodiments, the lung cancer is NSCLC.

### BRIEF DESCRIPTION OF THE FIGURES

FIG. 1 | The siRNA screen for drug tolerant persisters (DTPs) was initially developed and implemented using human non-small-cell lung cancer cell line PC9 using real-time confluence measurement (ESSEN Incucyte readout) and cell viability endpoint readout (CyQuant Direct cell proliferation assay). The screen was developed based on the survival and recovery of the DTPs after drug treatment. The screen consisted of three phases: Transfection, drug or media treatment and recovery phase. Final cell number was determined based on the Cyquant Direct cell proliferation assay signal.

FIG. 2A and FIG. 2B | The siRNA screen was run two times in completely independent conditions. For both siRNA screen run Z-factors were calculated based on the difference between the media and erlotinib treatment conditions for the non-targeting control (NTC) as well as between the non-targeting control and the positive control (HDAC3 siRNA single 3) in the erlotinib treatment condition. For both screens the Z-factor values comparing these conditions was between 0.5 and 1 insuring an excellent assay.

FIG. 3A, FIG. 3B, FIG. 3C and FIG. 3D | Cell number per well was normalized by the average cell number per well per plate for every condition in both media and erlotinib treatment. Correlation between duplicate well ran across two different plates was calculated.

FIG. 4A and FIG. 4B | For both siRNA screens Z-factors were calculated based on the difference between the media and erlotinib treatment conditions for the non-targeting control (NTC) as well as between the non-targeting control and the positive control (HDAC3 siRNA single 3) in the erlotinib treatment condition. For both screens the Z-factor values comparing these conditions was between 0.5 and 1 insuring an excellent assay.

FIG. 5A-1, FIG. 5A1-2, FIG. 5B-1, FIG. 5B1-2, FIG. 5C-1, FIG. 5C1-2, FIG. 5D-1, FIG. 5D1-2, FIG. 5E-1, FIG. 5E1-2, FIG. 5F-1, FIG. 5F1-2, FIG. 5G-1, FIG. 5G1-2, FIG. 5H-1, FIG. 5H1-2, FIG. 5I-1, FIG. 5I1-2, FIG. 5J-1, FIG. 5J1-2, FIG. 5K-1, FIG. 5K1-2, FIG. 5L-1, FIG. 5L1-2, FIG. 5M-1, FIG. 5M1-2, FIG. 5N-1, FIG. 5N1-2, FIG. 5O-1 and FIG. 5O1-2 | Cell number per well in siRNA screen run #1 and 2 for various chromatin modifiers identified as hits: A: ATRX, B: UBE2A, C:UBE2B, D:MYST4, E:EZH2, F:HDAC2, G:HDAC3, H:CDYL, I:LRWD1, J:CHD7, K:PHF10, L:PHF12, M:PHF23, N:CHD1, O:RING1B. Cell number in the media (Parental cells: diamond bars) and erlotinib (drug tolerant persisters (DTP): solid gray bars) conditions with 4 different specific siRNAs (Dharmacon siGenome siRNA) are presented along with the data for the non-targeting control (NTC).

FIG. 6 | Effect of siRNA knockdown of various essential components of the Polycomb group (PcG) multiprotein PRC1-like complex (PRC1): RING1B, CBX2, CBX3, CBX4, CBX6, CBX7, CBX8, on PC9 parental (solid gray bars: media condition) and DTP (diamond bars: erlotinib condition) cell number using 4 different specific siRNAs (Dharmacon siGenome siRNA) per gene. Data are presented along with the data for the non-targeting (NTC) and siTOX controls in similar treatment conditions.

FIG. 7 | Effect of siRNA knockdown of various component of the PRC1 and 2: EZH1, EZH2, SUZ12, EED, RBBP4, HDAC1, HDAC2, on PC9 parental (solid gray bars: media conditions) or DTP (diamond bars: erlotinib condition) cell number using 4 different specific siRNAs (Dharmacon siGenome siRNA) per gene. For the embryonic ectoderm development (EED) protein and HDAC1 8 different specific siRNAs (Dharmacon siGenome (×4) and On-Target Plus (OTP)(×4) siRNAs) were used. Data are presented along with the data for the non-targeting (NTC) and siTOX controls in similar treatment conditions.

FIG. 8A, FIG. 8B and FIG. 8C | Effect of siRNA knockdown of essential components of the PRC2 complex: EZH2 (A), SUZ12 (B) and EED (C) on PC9 parental (black bars: media conditions) or DTP (light gray bars: erlotinib condition) cell number using 8 different specific siRNAs (Dharmacon siGenome (×4) and On-Target Plus (OTP)(×4)) per gene. Data are presented along with the data for the non-targeting (NTC) and siTOX controls in similar treatment conditions.

FIG. 9A, FIG. 9B and FIG. 9C | Effect of siRNA knockdown of essential components of the PRC2 complex: EZH2 (A), SUZ12 (B) and EED (C) on H1299 parental (black bars: media conditions) or DTP (light gray bars: Paclitaxel condition) cell number using 8 different specific siRNAs (Dharmacon siGenome (×4) and On-Target Plus (OTP)(×4)) per gene. Data are presented along with the data for the non-targeting (NTC) and siTOX controls in similar treatment conditions.

FIG. 10A, FIG. 10B and FIG. 10C | Effect of siRNA knockdown of essential components of the PRC2 complex: EZH2 (A), SUZ12 (B) and EED (C) on PC9 drug tolerant established persisters (DTEP) treated with (light gray bars) or without erlotinib (black bars) using 8 different specific siRNAs (Dharmacon siGenome (×4) and On-Target Plus (OTP)(×4)) per gene. Data are presented along with the data for the non-targeting (NTC) and siTOX controls in similar treatment conditions.

FIG. 11A, FIG. 11B and FIG. 11C | Effect of 3-deazaneplanocin A (DZNep), a cyclopentenyl analog of 3-deazaadenosine previously described to deplete EZH2 levels and to inhibit trimethylation of lysine 27 on histone H3 in cultured human acute myeloid leukemia in a dose-dependent manner (0.2-1 μM) (Fiskus, W. et al. (2009) Blood 114(13), 2733-2743). DZNep was tested at concentrations ranging from 0.625 to 40 μM (A). PC9 cells were treated for 48 hours either with DZNep (DZNep/Media or DZNep/erlotinib) or DMSO control (DMSO/Media or DMSO/erlotinib) before treatment with (DTP) (DMSO/erlotinib or DZNep/erlotinib) or without erlotinib (parental)(DMSO/Media or DZNep/Media) for 72 hours. Cell number was determined after a 48-72 hours recovery phase in media alone. Effect of DZNep at 5 (B) and 0.625 μM (C) on PC9 parental (solid gray bars: media conditions) or DTP (diamond bars: erlotinib condition) cell number.

FIG. 12 | Effect of siRNA knockdown of essential components of the histone deacetylase NuRD complex (CHD4, MBD3, RBBP4, HDAC1, HDAC2) on PC9 parental (solid gray bars: media conditions) or DTP (diamond bars: erlotinib condition) cell number using 8 different specific siRNAs (Dharmacon siGenome (×4) and On-Target Plus (OTP)(×4)) per gene. Data are presented along with the data for the non-targeting (NTC) and siTOX controls in similar treatment conditions.

FIG. 13A and FIG. 13B | Effect of siRNA knockdown of ATRX (dark gray dots associated with words) compared to other chromatin modifiers (light gray dots) on H1299 parental (X axis: media conditions) or DTP (Y axis: Paclitaxel condition) Z score (calculated based on the NTC control across all screen plates after normalization to the data to NTC control per plate) using 4 different specific siRNAs (Dharmacon siGenome) per gene. Data are presented along with the data for the non-targeting (NTC)(dark gray dots to the right) and siTOX controls (dark grey dots to the left) in similar treatment conditions.

FIG. 14A and FIG. 14B | (A) Table of positive siRNAs identified in PC9 cells using erlotinib treatment. (B) Table of positive siRNAs identified in H1299 cells using paclitaxel treatment.

FIG. 15A, FIG. 15B and FIG. 15C | Histone H3K27me³ is increased while H3K27Ac is decreased in PC9 DTPs. (A) Schematic of histone H3 tail and amino acid positions of post-translational modification. The PRC2 complex which includes SUZ12, EZH2, and EED, methylates K27. (B) Histone H3K27me³ is increased while H3K27Ac is decreased in PC9 DTPs compared to the parental PC9 cell line as shown by Western blot. (C) Histone H3K27me³ is increased while H3K27Ac is decreased in PC9 DTPs compared to the parental PC9 cell line as measured by mass spectrometry.

FIG. 16A, FIG. 16B and FIG. 16C | DTPs in a variety of models display increased sensitivity to HDAC inhibitors. (A) SKBR3 treated with 2.5 Gy in media alone or presence of 25 nM of the HDAC inhibitor TSA. (B) SKBR3 treated with 10 Gy in media alone or presence of 25 nM of the HDAC inhibitor TSA. (C) SKBR3 treated with 1 μM Lapatinib in media alone or presence of 25 nM of the HDAC inhibitor TSA.

FIG. 17A, FIG. 17B and FIG. 17C | HDAC (1), 2 and 3 are involved in the establishment of drug tolerance. siRNA against HDAC2 and 3 as well as inhibitors that are HDAC1/2 or 3 biased disrupt the drug-tolerant state. (A-B) Effect of siRNA knockdown of essential components of HDAC2 and HDAC3 on PC9 parental (light grey bars: media conditions) or DTP (dark grey: erlotinib condition) cell number using 4 different specific siRNAs per gene. Data are presented along with the data for the non-targeting (NTC) and siTOX controls in similar treatment conditions. (C) Effect of (C1) G946, HDAC1/2 biased inhibitor, and (C2) G877, HDAC3 biased inhibitor. G946 and G877 were tested at concentrations 50 nM and 5 μM, respectively. PC9 cells were treated with (DTP) (DMSO/erlotinib or HDAC inhibitor/erlotinib) (C1 and C2) for 30 days or without erlotinib (parental) (DMSO/Media or HDAC inhibitor/Media) (data not shown). Erlotinib concentration 1 μM.

FIG. 18 | Effect of HDAC Inhibition using TSA (0.5 mg/kg) on erlotinib response in PC9 xenografts.

FIG. 19A and FIG. 19B | EZH2, a member of the PRC2, is involved in the establishment of drug-tolerance. Both siRNA against EZH2 or and small molecule inhibitors of EZH2 inhibitors (GSK126) disrupt the drug-tolerant state. (A) Effect of siRNA knockdown of EZH2 on PC9 parental (light grey bars: media conditions) or DTP (dark grey: erlotinib condition) cell number using 8 different specific siRNAs (Dharmacon siGenome (×4) and On-Target Plus (OTP)(×4)) per gene. Data are presented along with the data for the non-targeting (NTC) and siTOX controls in similar treatment conditions. (B) Effect of GSK126 was tested at a concentration 1 μM. PC9 cells were treated for 4 days either with GSK126 (with Media or with erlotinib) or DMSO control (DMSO/Media or DMSO/erlotinib) before treatment with (DTP) (DMSO/erlotinib or GSK126/erlotinib) (B, lower left and right respectively) for 30 days or without erlotinib (parental) (DMSO/Media or GSK126/Media) (B, upper left and right respectively).

FIG. 20A, FIG. 20B, FIG. 20C and FIG. 20D | EZH2 inhibitors (GSK126) disrupt the drug-tolerant state. (A) Increase in histone H3K27me³ in PC9 DTPs treated with erlotinib is inhibited by EZH2 small molecule inhibitor (GSK126) at 0.1, 0.5, and 2.5 μM as shown by Western blot. (B) nuc-red PC9 cells were treated with DMSO/erlotinib or GSK126/erlotinib at 0.1, 0.5, or 2.5 μM (dark grey bar) or without erlotinib (DMSO/Media or GSK126/Media) (light grey bar) and analyzed IncuCyte™ at day 9 and 3, respectively. (C) nuc-red PC9 cells were treated with DMSO/erlotinib or GSK126/erlotinib at 2.5 μM for 9 days and imaged. (D) nuc-red PC9 cells were treated with DMSO/erlotinib or GSK126/erlotinib at 2.5 μM for 30 days and stained with Giemsa. Erlotinib concentration 1 μM for FIG. 20A, FIG. 20B, FIG. 20C and FIG. 20D.

FIG. 21A and FIG. 21B | EZH2 inhibitors (GSK126 and EPZ-6438) disrupt the drug-tolerant state. (A) Increase in histone H3K27me³ in EVSAT DTPs treated with the PI3K inhibitor, GDC-0908, is inhibited by EZH2 small molecule inhibitor (GSK126 at 0.1, 0.5, and 2.5 μM and EPZ-6438 at 0.05, 0.1, and 0.5 μM) as shown by Western blot. (B) nuc-red EVSAT cells were treated with DMSO/GDC-0908, GSK126/GDC-0908 at 0.1, 0.5, or 2.5 μM, or EPZ-6438/GDC-0908 at 0.05, 0.1, and 0.5 μM (dark grey bar) or without GDC-0908 (DMSO/Media, GSK126/Media, EPZ-6438/Media) (light grey bar) and analyzed IncuCyte™ at day 9 and 3, respectively. GDC-0908 concentration 2 μM for FIG. 21A and FIG. 21B.

FIG. 22A, FIG. 22B, FIG. 22C and FIG. 22D | EZH2 inhibitor (EPZ-6438) disrupts the drug-tolerant state. (A) Increase in histone H3K27me³ in PC9 DTPs treated with erlotinib is inhibited by EZH2 small molecule inhibitor (EPZ-6438) at 0.05, 0.1, 0.5, 1, and 1.5 μM as shown by Western blot. (B) nuc-red PC9 cells were treated with DMSO/erlotinib or EPZ-6438/erlotinib at 0.05, 0.1, 0.5, 1, and 1.5 μM (dark grey bar) or without erlotinib (DMSO/Media or EPZ-6438/Media) (light grey bar) and analyzed IncuCyte™ at day 9 and 3, respectively. (C) nuc-red PC9 cells were treated with DMSO/erlotinib or EPZ-6438/erlotinib at 1 μM for 9 days and imaged. (D) nuc-red PC9 cells were treated with DMSO/erlotinib or EPZ-64386/erlotinib at 0.05, 0.1, or 1.0 μM) for 30 days and stained with Giemsa. Erlotinib concentration 1 μM for FIG. 22A, FIG. 22B, FIG. 22C and FIG. 22D.

### DETAILED DESCRIPTION

### I. Definitions

An “antagonist” (interchangeably termed “inhibitor”) of a polypeptide of interest is an agent that interferes with activation or function of the polypeptide of interest, e.g., partially or fully blocks, inhibits, or neutralizes a biological activity mediated by a polypeptide of interest. For example, an antagonist of polypeptide X may refers to any molecule that partially or fully blocks, inhibits, or neutralizes a biological activity mediated by polypeptide X. Examples of inhibitors include antibodies; ligand antibodies; small molecule antagonists; antisense and inhibitory RNA (e.g., shRNA) molecules. Preferably, the inhibitor is an antibody or small molecule which binds to the polypeptide of interest. In a particular embodiment, an inhibitor has a binding affinity (dissociation constant) to the polypeptide of interest of about 1,000 nM or less. In another embodiment, inhibitor has a binding affinity to the polypeptide of interest of about 100 nM or less. In another embodiment, an inhibitor has a binding affinity to the polypeptide of interest of about 50 nM or less. In a particular embodiment, an inhibitor is covalently bound to the polypeptide of interest. In a particular embodiment, an inhibitor inhibits signaling of the polypeptide of interest with an IC₅₀ of 1,000 nM or less. In another embodiment, an inhibitor inhibits signaling of the polypeptide of interest with an IC₅₀ of 500 nM or less. In another embodiment, an inhibitor inhibits signaling of the polypeptide of interest with an IC₅₀ of 50 nM or less. In certain embodiments, the antagonist reduces or inhibits, by at least 10%, 20%, 30%, 40%, 50%, 60%, 70%, 80%, 90% or more, the expression level or biological activity of the polypeptide of interest. In some embodiments, the polypeptide of interest is a chromatin modifier. In some embodiments, the polypeptide of interest is EGFR.

The term “polypeptide” as used herein, refers to any native polypeptide of interest from any vertebrate source, including mammals such as primates (e.g., humans) and rodents (e.g., mice and rats), unless otherwise indicated. The term encompasses “full-length,” unprocessed polypeptide as well as any form of the polypeptide that results from processing in the cell. The term also encompasses naturally occurring variants of the polypeptide, e.g., splice variants or allelic variants.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “small molecule” refers to any molecule with a molecular weight of about 2000 daltons or less, preferably of about 500 daltons or less.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms anti-polypeptide of interest antibody and “an antibody that binds to” a polypeptide of interest refer to an antibody that is capable of binding a polypeptide of interest with sufficient affinity such that the antibody is useful as a diagnostic and/or therapeutic agent in targeting a polypeptide of interest. In one embodiment, the extent of binding of an anti-polypeptide of interest antibody to an unrelated, non-polypeptide of interest protein is less than about 10% of the binding of the antibody to a polypeptide of interest as measured, e.g., by a radioimmunoassay (RIA). In certain embodiments, an antibody that binds to a polypeptide of interest has a dissociation constant (Kd) of ≦1 μM, ≦100 nM, ≦10 nM, ≦1 nM, ≦0.1 nM, ≦0.01 nM, or ≦0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-polypeptide of interest antibody binds to an epitope of a polypeptide of interest that is conserved among polypeptides of interest from different species. In some embodiments, the polypeptide of interest is a chromatin modifier. In some embodiments, the polypeptide of interest is EGFR.

A “blocking antibody” or an “antagonist antibody” is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “chimeric” antibody refers to an antibody in which a portion of the heavy and/or light chain is derived from a particular source or species, while the remainder of the heavy and/or light chain is derived from a different source or species.

The terms “full length antibody,” “intact antibody,” and “whole antibody” are used herein interchangeably to refer to an antibody having a structure substantially similar to a native antibody structure or having heavy chains that contain an Fc region.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody conjugated to one or more heterologous molecule(s), including but not limited to a cytotoxic agent.

As used herein, the term “targeted therapeutic” refers to a therapeutic agent that binds to polypeptide(s) of interest and inhibits the activity and/or activation of the specific polypeptide(s) of interest. Examples of such agents include antibodies and small molecules that bind to the polypeptide of interest.

A “chemotherapy” refers to a chemical compound useful in the treatment of cancer. Examples of chemotherapies include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANEn), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylomithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu), chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents), growth inhibitory agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

“Individual response” or “response” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metasisis; (5) relief, to some extent, of one or more symptoms associated with the disease or disorder (e.g., cancer); (6) increase in the length of progression free survival; and/or (7) decreased mortality at a given point of time following treatment.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression). The difference between said two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

An “effective amount” of a substance/molecule, e.g., pharmaceutical composition, refers to an amount effective, at dosages and for periods of time necessary, to achieve the desired therapeutic or prophylactic result.

A “therapeutically effective amount” of a substance/molecule may vary according to factors such as the disease state, age, sex, and weight of the individual, and the ability of the substance/molecule to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The phrase “pharmaceutically acceptable salt” as used herein, refers to pharmaceutically acceptable organic or inorganic salts of a compound.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concomitantly” is used herein to refer to administration of two or more therapeutic agents, give in close enough temporal proximity where their individual therapeutic effects overlap in time. Accordingly, concurrent administration includes a dosing regimen when the administration of one or more agent(s) continues after discontinuing the administration of one or more other agent(s). In some embodiments, the concomitantly administration is concurrently, sequentially, and/or simultaneously.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

As is understood by one skilled in the art, reference to “about” a value or parameter herein includes (and describes) embodiments that are directed to that value or parameter per se. For example, description referring to “about X” includes description of “X”.

It is understood that aspect and embodiments of the invention described herein include “consisting” and/or “consisting essentially of” aspects and embodiments. As used herein, the singular form “a”, “an”, and “the” includes plural references unless indicated otherwise.

### II. Methods and Uses

Provided herein are methods of using modulators of chromatin modifiers, for example, for treating cancer and preventing drug resistance. In some embodiments, the individual is selected for treatment with a cancer therapy agent (e.g., targeted therapies, chemotherapies, and/or radiotherapies). In some embodiments, the individual starts treatment comprising administration of the modulator of the chromatin modifier prior to treatment with the cancer therapy agent. In some embodiments, the individual concurrently receives treatment comprising the modulator of the chromatin modifier and the cancer therapy agent. In some embodiments, the modulator of the chromatin modifier increases period of cancer sensitivity and/or delay development of cancer resistance. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Also provided herein are methods utilizing a modulator of a chromatin modifier and a cancer therapy agent (e.g., targeted therapy, chemotherapy, and/or radiotherapy). In some embodiments, the modulator of the chromatin modifier is an antagonist of a chromatin modifier. In some embodiments, the cancer therapy agent is an EGFR antagonist or a taxane (e.g., paclitaxel).

In particular, provided herein are methods of treating cancer in an individual comprising administering to the individual (a) a modulator of a chromatin modifier and (b) a cancer therapy agent (e.g., targeted therapy, chemotherapy, and/or radiotherapy). In some embodiments, the respective amounts of the modulator of the chromatin modifier and the cancer therapy agent (e.g., the targeted therapy, chemotherapy, and/or radiotherapy) are effective to increase the period of cancer sensitivity and/or delay the development of cell resistance to the cancer therapy agent (e.g., the targeted therapy, chemotherapy, and/or radiotherapy). In some embodiments, the respective amounts of the modulator of the chromatin modifier and the cancer therapy agent (e.g., the targeted therapy, chemotherapy, and/or radiotherapy) are effective to increase efficacy of a cancer treatment comprising the cancer therapy agent (e.g., the targeted therapy, chemotherapy, and/or radiotherapy). For example, in some embodiments, the respective amounts of the modulator of the chromatin modifier and the cancer therapy agent (e.g., the targeted therapy, chemotherapy, and/or radiotherapy) are effective to increased efficacy compared to a standard treatment comprising administering an effective amount of cancer therapy agent (e.g., targeted therapy, chemotherapy, and/or radiotherapy) without (in the absence of) the modulator of the chromatin modifier. In some embodiments, the respective amounts of the modulator of the chromatin modifier and the cancer therapy agent (e.g., the targeted therapy, chemotherapy, and/or radiotherapy) are effective to increased response (e.g., complete response) compared to a standard treatment comprising administering an effective amount of cancer therapy agent (e.g., targeted therapy, chemotherapy, and/or radiotherapy) without (in the absence of) the modulator of the chromatin modifier. In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Further provided herein are methods of increasing efficacy of a cancer treatment comprising a cancer therapy agent (e.g., targeted therapy, chemotherapy, and/or radiotherapy) in an individual comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Provided herein of treating cancer in an individual wherein cancer treatment comprising administering to the individual (a) an effective amount of a modulator of a chromatin modifier and (b) an effective amount of a cancer therapy agent (e.g., targeted therapy, chemotherapy, and/or radiotherapy), wherein the cancer treatment has increased efficacy compared to a standard treatment comprising administering an effective amount of cancer therapy agent (e.g., targeted therapy, chemotherapy, and/or radiotherapy) without (in the absence of) the modulator of the chromatin modifier. In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

In addition, provided herein are methods of delaying and/or preventing development of cancer resistant to a cancer therapy agent (e.g., targeted therapy, chemotherapy, and/or radiotherapy) in an individual, comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Provided herein are methods of treating an individual with cancer who has increased likelihood of developing resistance to a cancer therapy agent (e.g., targeted therapy, chemotherapy, and/or radiotherapy) comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Further provided herein are methods of increasing sensitivity to a cancer therapy agent (e.g., targeted therapy, chemotherapy, and/or radiotherapy) in an individual with cancer comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

In addition, provided herein are methods of extending the period of a cancer therapy agent (e.g., targeted therapy, chemotherapy, and/or radiotherapy) sensitivity in an individual with cancer comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

Provided herein are also methods of extending the duration of response to a cancer therapy agent (e.g., targeted therapy, chemotherapy, and/or radiotherapy) in an individual with cancer comprising administering to the individual (a) an effective amount of a modulator of a chromatin modifier and (b) an effective amount of the cancer therapy agent (e.g., the targeted therapy, chemotherapy, and/or radiotherapy). In some embodiments, the modulator of the chromatin modifier and the cancer therapy agent is administered concomitantly. In some embodiments, the cancer therapy agent is an EGFR antagonist. In some embodiments, the combination therapy comprises (a) a modulator of a chromatin modifier (e.g., an antagonist of a chromatin modifier) and (b) an EGFR antagonist. In some embodiments, the EGFR antagonist is erlotinib and/or gefitinib. In some embodiments, the cancer therapy agent is a taxane. In some embodiments, the combination therapy comprises (a) a modulator of a chromatin modifier (e.g., an antagonist of a chromatin modifier) and (b) a taxane (e.g., paclitaxel). In some embodiments, the taxane is paclitaxel. In some embodiments, the modulator of the chromatin modifier is an antagonist of a chromatin modifier. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

In addition to providing improved treatment for cancer, administration of certain combinations described herein may improve the quality of life for a patient compared to the quality of life experienced by the same patient receiving a different treatment. For example, administration of a combination of the antagonist of a chromatin modifier and the cancer therapy agent (e.g., the targeted therapy, chemotherapy, and/or radiotherapy), as described herein to an individual may provide an improved quality of life compared to the quality of life the same patient would experience if they received only cancer therapy agent (e.g., targeted therapy, chemotherapy, and/or radiotherapy) as therapy. For example, the combined therapy with the combination described herein may lower the dose of cancer therapy agent (e.g., targeted therapy, chemotherapy, and/or radiotherapy) needed, thereby lessening the side-effects associated with the therapeutic (e.g. nausea, vomiting, hair loss, rash, decreased appetite, weight loss, etc.). The combination may also cause reduced tumor burden and the associated adverse events, such as pain, organ dysfunction, weight loss, etc. Accordingly, one aspect provides antagonist of a chromatin modifier for therapeutic use for improving the quality of life of a patient treated for a cancer with a cancer therapy agent (e.g., targeted therapy, chemotherapy, and/or radiotherapy). In some embodiments, the targeted therapy is an EGFR antagonist. In some embodiments, the EGFR antagonist is erlotinib and/or gefitinib. In some embodiments, the chemotherapy comprises a taxane. In some embodiments, the taxane is paclitaxel. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

In some embodiments of any of the methods, the modulator of a chromatin modifier is an antibody, binding polypeptide, binding small molecule, or polynucleotide. In some embodiments of any of the methods, the antagonist of a chromatin modifier is an antibody, binding polypeptide, binding small molecule, or polynucleotide. In some embodiments, the antagonist of a chromatin modifier is an antagonist of EZH2, SUZ12, and/or EED. In some embodiments, the antagonist of a chromatin modifier is an antagonist of HDAC2 and/or HDAC3.

In some embodiments of any of the methods, the cancer therapy agent is a targeted therapy. In some embodiments of any of the methods, the cancer therapy agent is a chemotherapy. In some embodiments of any of the methods, the cancer therapy agent is a radiotherapy.

Cancer having resistance to a therapy as used herein includes a cancer which is not responsive and/or reduced ability of producing a significant response (e.g., partial response and/or complete response) to the therapy. Resistance may be acquired resistance which arises in the course of a treatment method. In some embodiments, the acquired drug resistance is transient and/or reversible drug tolerance. Transient and/or reversible drug resistance to a therapy includes wherein the drug resistance is capable of regaining sensitivity to the therapy after a break in the treatment method. In some embodiments, the acquired resistance is permanent resistance. Permanent resistance to a therapy includes a genetic change conferring drug resistance.

Cancer having sensitivity to a therapy as used herein includes cancer which is responsive and/or capable of producing a significant response (e.g., partial response and/or complete response).

Methods of determining of assessing acquisition of resistance and/or maintenance of sensitivity to a therapy are known in the art and described in the Examples. Drug resistance and/or sensitivity may be determined by (a) exposing a reference cancer cell or cell population to a cancer therapy agent in the presence and/or absence of a modulator of a chromatin modifier (e.g., an antagonist of a chromatin modifier) and/or (b) assaying, for example, for one or more of cancer cell growth, cell viability, level and/or percentage apoptosis, and/or response. Drug resistance and/or sensitivity may be measured over time and/or at various concentrations of cancer therapy agent and/or amount of antagonist of a chromatin modifier. Drug resistance and/or sensitivity further may be measured and/or compared to a reference cell line (e.g., PC9 and/or H1299) including parental cells, drug tolerant persister cells, and/or drug tolerant expanded persister cells of the cell line. In some embodiments, cell viability may be assayed by CyQuant Direct cell proliferation assay. Changes in acquisition of resistance and/or maintenance of sensitivity such as drug tolerance may be assessed by assaying the growth of drug tolerant persisters as described in the Examples and Sharma et al. Changes in acquisition of resistance and/or maintenance of sensitivity such as permanent resistance and/or expanded resisters may be assessed by assaying the growth of drug tolerant expanded persisters as described in the Examples and Sharma et al. In some embodiments, resistance may be indicated by a change in IC₅₀, EC₅₀ or decrease in tumor growth in drug tolerant persisters and/or drug tolerant expanded persisters. In some embodiments, the change is greater than about any of 50%, 100%, and/or 200%. In addition, changes in acquisition of resistance and/or maintenance of sensitivity may be assessed in vivo for examples by assessing response, duration of response, and/or time to progression to a therapy, e.g., partial response and complete response. Changes in acquisition of resistance and/or maintenance of sensitivity may be based on changes in response, duration of response, and/or time to progression to a therapy in a population of individuals, e.g., number of partial responses and complete responses.

In some embodiments of any of the methods, the cancer is a solid tumor cancer. In some embodiments, the cancer is lung cancer, breast cancer, colorectal cancer, colon cancer, melanoma, and/or pancreatic cancer. In some embodiments, the cancer is colorectal cancer. In some embodiments, the cancer is lung cancer (e.g., non-small cell lung cancer (NSCLC)). In some embodiments, the cancer is pancreatic cancer. In some embodiments, the cancer is breast cancer. In some embodiments, the cancer is melanoma. In some embodiments, the cancer is CD133 positive. In some embodiments, the cancer is CD24 positive. In some embodiments, the cancer has high levels of H3K27 trimethylation. In some embodiments, the cancer is at risk of developing increasing levels of H3K27 trimethylation. In some embodiments, the cancer has low levels of H3K27 acetylation. In some embodiments, the cancer is at risk of developing decreasing levels of H3K27 acetylation.

The cancer in any of the combination therapies methods described herein when starting the method of treatment comprising the antagonist of a chromatin modifier and the cancer therapy agent (e.g., the targeted therapy, chemotherapy, and/or radiotherapy) may be sensitive (examples of sensitive include, but are not limited to, responsive and/or capable of producing a significant response (e.g., partial response and/or complete response)) to a method of treatment comprising the cancer therapy agent (e.g., the targeted therapy, chemotherapy, and/or radiotherapy) alone. The cancer in any of the combination therapies methods described herein when starting the method of treatment comprising the antagonist of a chromatin modifier and the cancer therapy agent (e.g., the targeted therapy, chemotherapy, and/or radiotherapy) may not be resistant (examples of resistance include, but are not limited to, not responsive and/or reduced ability and/or incapable of producing a significant response (e.g., partial response and/or complete response) to a method of treatment comprising the cancer therapy agent (e.g., the targeted therapy, chemotherapy, and/or radiotherapy) alone. In some embodiments, the cancer therapy agent is a targeted therapy and the targeted therapy is an antagonist of EGFR. In some embodiments, the cancer therapy agent is a chemotherapy and the chemotherapy is a taxane.

In some embodiments of any of the methods, the individual according to any of the above embodiments may be a human.

In some embodiments of any of the methods, the combination therapy may be concomitantly administered. In some embodiments of any of the methods, the combination therapies may encompass combined administration (where two or more therapeutic agents are included in the same or separate formulations), and separate administration, in which case, administration of the antagonist of a chromatin modifier and the cancer therapy agent (e.g., the targeted therapy, chemotherapy, and/or radiotherapy) can occur prior to, simultaneously, sequentially, concurrently, and/or following, administration of the additional therapeutic agent and/or adjuvant. In some embodiments of any of the methods, the chromatin modifier is administered prior to and/or concurrently with the cancer therapy agent (e.g., the targeted therapy, chemotherapy, and/or radiotherapy). In some embodiments, the combination therapy further comprises radiation therapy and/or additional therapeutic agents.

In some embodiments of any of the methods, the modulator of the chromatin modifier (e.g., antagonist of chromatin modifier) and the cancer therapy agent (e.g., the targeted therapy and/or chemotherapy, and/or radiotherapy) can be administered by any suitable means, including oral, parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some embodiments of any of the methods, the modulator of the chromatin modifier (e.g., antagonist of chromatin modifier) and the cancer therapy agent (e.g., the targeted therapy, chemotherapy, and/or radiotherapy) described herein may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antagonist of a chromatin modifier and the cancer therapy agent (e.g., the targeted therapy, chemotherapy, and/or radiotherapy) need not be, but is optionally formulated with one or more agents currently used to prevent or treat the disorder in question. The effective amount of such other agents depends on the amount of the antagonist of a chromatin modifier and the cancer therapy agent (e.g., the targeted therapy, chemotherapy, and/or radiotherapy) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a modulator of a chromatin modifier (e.g., an antagonist of a chromatin modifier) and the cancer therapy agent (e.g., the targeted therapy, chemotherapy, and/or radiotherapy) described herein (when used alone or in combination with one or more other additional therapeutic agents) will depend on the type of disease to be treated, the severity and course of the disease, whether the antagonist of a chromatin modifier and the cancer therapy agent (e.g., the targeted therapy, chemotherapy, and/or radiotherapy) is administered for preventive or therapeutic purposes, previous therapy, the patient's clinical history and response to the antagonist of a chromatin modifier and the cancer therapy agent (e.g., the targeted therapy, chemotherapy, and/or radiotherapy), and the discretion of the attending physician. The antagonist of a chromatin modifier and the cancer therapy agent (e.g., the targeted therapy, chemotherapy, and/or radiotherapy) is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives from about two to about twenty, or e.g., about six doses of the antagonist of a chromatin modifier and the cancer therapy agent (e.g., the targeted therapy, chemotherapy, and/or radiotherapy)). An initial higher loading dose, followed by one or more lower doses may be administered. An exemplary dosing regimen comprises administering. However, other dosage regimens may be useful. The progress of this therapy is easily monitored by conventional techniques and assays. In some embodiments, the combination therapy comprises (a) a modulator of a chromatin modifier (e.g., an antagonist of a chromatin modifier) and (b) taxane (e.g., paclitaxel). In some embodiments, the combination therapy comprises (a) a modulator of a chromatin modifier (e.g., an antagonist of a chromatin modifier) and (b) EGFR antagonist.

It is understood that any of the above formulations or therapeutic methods may be carried out using an immunoconjugate as the chromatin modifier and/or EGFR antagonist.

### III. Therapeutic Compositions

Provided herein are modulator of the chromatin modifier (e.g., antagonist of chromatin modifier) and cancer therapy agent (e.g., the targeted therapy, chemotherapy, and/or radiotherapy) for use in the methods described herein. In certain embodiments, the combination increases the efficacy of the cancer therapy agent (e.g., the targeted therapy, chemotherapy, and/or radiotherapy) administered alone. In certain embodiments, the combination delays and/or prevents development of cancer resistance to the cancer therapy agent (e.g., the targeted therapy, chemotherapy, and/or radiotherapy). In certain embodiments, the combination extends the period of the cancer therapy agent (e.g., the targeted therapy, chemotherapy, and/or radiotherapy) sensitivity in an individual with cancer. In some embodiments, the antagonists of a chromatin modifier and/or the cancer therapy agents (e.g., the targeted therapies) are an antibody, binding polypeptide, binding small molecule, and/or polynucleotide.

In some embodiments of any of the methods, the modulator of the chromatin modifier is an antagonist of a chromatin modifier.

In some embodiments of any of the methods, the chromatin modifier is a member of polycomb repressive complex (PRC). In some embodiments, the member of PRC is a member of polycomb repressive complex 1 (PRC1). In some embodiments, the member of PRC1 is one or more of RING1B, CBX3, CBX6, and CBX8. In some embodiments, the member of PRC is a member of polycomb repressive complex 2 (PRC2). In some embodiments, the member of PRC2 is EZH2 and/or EED. In some embodiments, the member of the PRC2 is EZH2.

In some embodiments of any of the methods, the chromatin modifier is a member of nucleosome remodeling and deacetylation complex (NuRD). In some embodiments, the member of NuRD is one or more of CHD4, RBBP4, HDAC1, HDAC2, and HDAC3. In some embodiments, the member of NuRD is HDAC2 and/or HDAC3.

In some embodiments of any of the methods, the chromatin modifier is an ubiquitin-conjugating enzyme. In some embodiments, the ubiquitin-conjugating enzyme is UBE2A and/or UBE2B.

In some embodiments of any of the methods, the chromatin modifier is one or more of ATRX, MYST4, CDYL, LRWD1, CHD7, PHF10, PHF12, PHF23, CHD1, MGEA5, MLLT10, SIRT4, TP53BP1, BRDT, CBX6, EVI1, GTF3C4, HIRA, MPHOSPH8, NCOA1, RBBP5, TDRD7, and ZCWPW1. In some embodiments of any of the methods, the chromatin modifier is one or more of ATRX, MYST4, CDYL, LRWD1, CHD7, PHF10, PHF12, PHF23, and CHD1. In some embodiments of any of the methods, the chromatin modifier is one or more of MGEA5, MLLT10, SIRT4, TP53BP1, ATRX, BRDT, CBX6, CHD1, EVI1, GTF3C4, HIRA, MPHOSPH8, NCOA1, RBBP5, TDRD7, and ZCWPW1.

Amino acid sequences of various human chromatin modifiers are known in the art and are publicly available. See e.g., ATRX (e.g., Entrez ID 546; UniProtBD/Swiss-Prot P46100-1, P46100-2, P46100-3, P46100-4, P46100-5, and/or P46100-6), UBE2A (e.g., Entrez ID 7319; UniProtBD/Swiss-Prot 49459-1, P49459-2, and/or P49459-3), UBE2B (e.g., Entrez ID 7320; UniProtBD/Swiss-Prot P63146), MYST4 (e.g., Entrez ID 23522; UniProtBD/Swiss-Prot Q8WYB5-1, Q8WYB5-2, and/or Q8WYB5-3), EZH2 (e.g., Entrez ID 2146; UniProtBD/Swiss-Prot Q15910-1, Q15910-2, Q15910-3, Q15910-4, and/or Q15910-5), HDAC2 (e.g., Entrez ID 3066; UniProtBD/Swiss-Prot Q92769), HDAC3 (e.g., Entrez ID 8841; UniProtBD/Swiss-Prot 015379-1 and/or O15379-2), CDYL (e.g., Entrez ID 9425; UniProtBD/Swiss-Prot Q9Y232-1, Q9Y232-2, Q9Y232-3, and/or Q9Y232-4), LRWD1 (e.g., Entrez ID 222229; UniProtKB/Swiss-Prot Q9UFCO), CHD7 (e.g., Entrez ID 55636; UniProtBD/Swiss-Prot Q9P2D1-1 and/or Q9P2D1-2), PHF10 (e.g., Entrez ID 55274; UniProtBD/Swiss-Prot Q8WUB8-1, Q8WUB8-2, and/or Q8WUB8-3), PHF12 (e.g., Entrez ID 57649; UniProtBD/Swiss-Prot Q96QT6-1, Q96QT6-2, Q96QT6-3, and/or Q96QT6-4), PHF23 (e.g., Entrez ID 79142; UniProtBD/Swiss-Prot Q9BUL5-1, and/or Q9BUL5-2), CHD1 (Entrez ID 1105; UniProtKB/Swiss-Prot O14646-1 and/or O14646-2), RING1B (e.g., Entrez ID 6045; UniProtBD/Swiss-Prot Q99496), EED (e.g., Entrez ID 8726; UniProtBD/Swiss-Prot O75530-1, 075530-2, and/or O75530-3), CBX3 (e.g., Entrez ID 11335; UniProtBD/Swiss-Prot Q13185), CBX6 (e.g., Entrez ID 23466; UniProtBD/Swiss-Prot O95503), CBX8 (e.g., Entrez ID 57332; UniProtBD/Swiss-Prot Q9HC52), CHD4 (e.g., Entrez ID 1108; UniProtBD/Swiss-Prot Q14839-1 and/or Q14839-2), RBBP4 (e.g., Entrez ID 5928; UniProtBD/Swiss-Prot Q09028-1, Q09028-2, Q09028-3, and/or Q09028-4), MGEA5 (e.g., UniProtBD/Swiss-Prot B4DYV7), MLLT10 (e.g., UniProtBD/Swiss-Prot P55197-1, P55197-2, and/or P55197-3), SIRT4 (e.g., UniProtBD/Swiss-Prot Q9Y6E7), TP53BP1 (e.g., UniProtBD/Swiss-Prot Q12888-1 and/or Q12888-2), BRDT (e.g., UniProtBD/Swiss-Prot Q58F21-1, Q58F21-2, Q58F21-3, Q58F21-4, and/or Q58F21-5), GTF3C4 (e.g., UniProtBD/Swiss-Prot Q05CN7), EVI (e.g., UniProtBD/Swiss-Prot Q9UBK3), HIRA (e.g., UniProtBD/Swiss-Prot P54198-1 and/or P54198-2), MPHOSPH8 (e.g., UniProtBD/Swiss-Prot Q99549-1 and/or Q99549-2), NCOA1 (e.g., UniProtBD/Swiss-Prot Q15788-1, Q15788-2, and/or Q15788-3), RBBP5 (e.g., UniProtBD/Swiss-Prot Q15291-1 and/or Q15291-2), TDRD7 (e.g., UniProtBD/Swiss-Prot Q8NHU6-1, Q8NHU6-2, and/or Q8NHU6-3), and/or ZCWPW1 (e.g., UniProtBD/Swiss-Prot Q9HOM4-1, Q9HOM4-2, Q9HOM4-3, Q9HOM4-4, and/or Q9HOM4-5).

Examples of EZH2 inhibitors include antibodies as described in WO1996/035784, binding polypeptides as described in WO2004/052392, binding small molecules in WO2011/140325, WO2011/140324, WO2012/034132, WO2012/005805, WO2013049770, U.S. Pat. No. 8,410,088, US20120071418, WO2012050532, WO2007149782, Verma et al. ACS Med. Chem. Lett. 3(12): 1091-1096 (2012), polynucleotides as described in WO2011/111072, WO2012/050532, WO2003/070887, and/or generally in WO2009/006577, WO2011/103016, WO2005/034845, which are all incorporated by reference in their entirety. In some embodiments, the EZH2 inhibitor inhibits histone H3 K27 tri-methylation. In some embodiments, the EZH2 inhibitor reduces histone H3 K27 tri-methylation. In some embodiments, the EZH2 inhibitor increases one or more of histone H3 K27 di, mono, and/or un-methylation. In some embodiments, the EZH2 inhibitor results in an increase in histone H3 K27 acetylation. In some embodiments, the EZH2 inhibitor is Isoliquiritigenin. In some embodiments, the EZH2 inhibitor is DZNeP and/or pharmaceutical acceptable salts and/or derivatives thereof. In some embodiments, the EZH2 inhibitor is GSK343 and/or pharmaceutical acceptable salts and/or derivatives thereof.

In some embodiments, the EZH2 inhibitor is GSK126 and/or pharmaceutical acceptable salts and/or derivatives thereof (GSK126 described in McCabe et al. Nature 492:108-112 (2012)). In some embodiments, the EZH2 inhibitor is CAS #1346574-57-9 or pharmaceutically acceptable salt thereof. In some embodiments, the EZH2 inhibitor is (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide or a pharmaceutically acceptable salt thereof. In some embodiments, the EZH2 inhibitor is

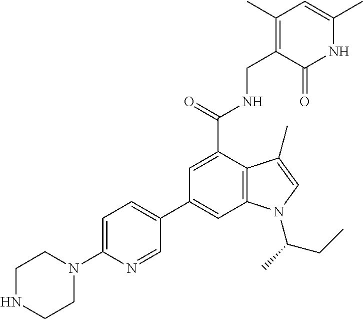 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt thereof.

In some embodiments, the EZH2 inhibitor is GSK926 and/or pharmaceutical acceptable salts and/or derivatives thereof.

In some embodiments, the EZH2 inhibitor is a compound of formula (I):

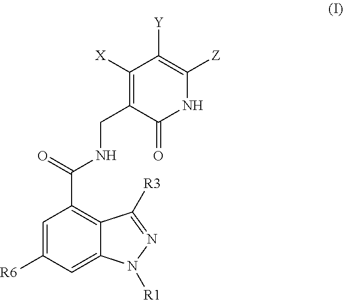

wherein

X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted a substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};

Y is H or halo;

R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};

R³ is hydrogen, (C₁-C₈)alkyl, cyano, trifluoromethyl, NR^{a}R^{b}, or halo;

R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalky unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆—C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};

R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl) ((C₁-C₄)alkyl);

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

each R^{c} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};

or a salt thereof.

In some embodiments, the EZH2 inhibitor is a compound of formula (II):

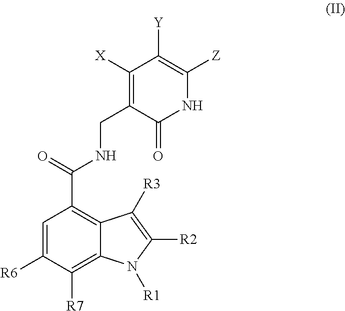

wherein

X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, NR^{a}NR^{a}C(O)OR^{a}, —OR^{a}, —OC(O)R^{a}, and —OC(O)NR^{a}R^{b};

Y is H or halo;

R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};

R² is hydrogen, (C₁-C₈)alkyl, trifluoromethyl, alkoxy, or halo, in which said (C₁-C₈)alkyl maybe substituted with one to two groups selected from: amino, and (C₁-C₃)alkylamino;

R⁷ is hydrogen, (C₁-C₃)alkyl, or alkoxy; R³ is hydrogen, (C₁-C₈)alkyl, cyano, trifluoromethyl, —NR^{a}R^{b}, or halo;

R⁶ is selected from the group consisting of hydrogen, halo, (C₁-C₈)alkyl, (C₂-C₈)alkenyl, —B(OH)₂, substituted or unsubstituted (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C₁-C₆)alkyl(R^{c})₁₋₂, —S(C₁-C₆)alkyl(R^{c})₁₋₂, —(C₁-C₆)alkyl(R^{c})₁₋₂, (C₁-C₈)alkyl-heterocycloalkyl, (C₃-C₈)cycloalkyl-heterocycloalkyl, halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, SOR^{a}, SO₂R^{a}, SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};

each R^{c} is independently (C₁-C₄)alkylamino, —NR^{a}SO₂R^{b}, —SOR^{a}, —SO₂R^{a}, —NR^{a}C(O)OR^{a}, —NR^{a}R^{b}, or —CO₂R^{a};

R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

or a salt thereof.

In some embodiments, the EZH2 inhibitor is S-adenosyl-L-homocysteine or a pharmaceutically acceptable salt thereof and/or

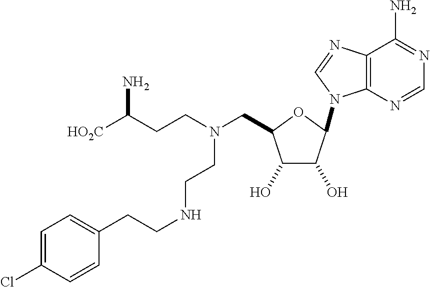

or a pharmaceutical acceptable salt thereof.

In some embodiments, the EZH2 inhibitor is a compound of formula (III)

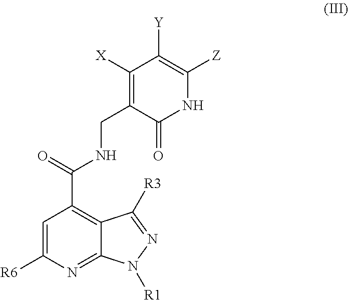

wherein

X and Z are selected independently from the group consisting of hydrogen, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, (C₆-C₁₀)bicycloalkyl, unsubstituted or substituted heterocycloalkyl, unsubstituted or substituted heterocycloalkyl-(C₁-C₈)alkyl or —(C₂-C₈)alkenyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, halo, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, and —OC(O)NR^{a}R^{b};

Y is H or halo;

R¹ is (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl or —(C₂-C₈)alkenyl, unsubstituted or substituted (C₅-C₈)cycloalkenyl, unsubstituted or substituted (C₅-C₈)cycloalkenyl-(C₁-C₈)alkyl or —(C₂-C₈)alkenyl, unsubstituted or substituted (C₆-C₁₀)bicycloalkyl, unsubstituted or substituted heterocycloalkyl or —(C₂-C₈)alkenyl, unsubstituted or substituted heterocycloalkyl-(C₁-C₈)alkyl, unsubstituted or substituted aryl, unsubstituted or substituted aryl-(C₁-C₈)alkyl or —(C₂-C₈)alkenyl, unsubstituted or substituted heteroaryl, unsubstituted or substituted heteroaryl-(C₁-C₈)alkyl or —(C₂-C₈)alkenyl, —COR^{a}, —CO₂R^{a}, —CONR^{a}R^{b}, —CONR^{a}NR^{a}R^{b};

R³ is hydrogen, (C₁-C₈)alkyl, cyano, trifluoromethyl, —NR^{a}R^{b}, or halo;

R⁶ is selected from the group consisting of hydrogen, halo, (C₁-C₈)alkyl, (C₂-C₈)alkenyl, (C₂-C₈)alkynyl, unsubstituted or substituted (C₃-C₈)cycloalkyl, unsubstituted or substituted (C₃-C₈)cycloalkyl-(C₁-C₈)alkyl, unsubstituted or substituted (C₅-C₈)cycloalkenyl, unsubstituted or substituted (C₅-C₈)cycloalkenyl-(C₁-C₈)alkyl, (C₆-C₁₀)bicycloalkyl, unsubstituted or substituted heterocycloalkyl, unsubstituted or substituted heterocycloalkyl-(C₁-C₈)alkyl, unsubstituted or substituted aryl, unsubstituted or substituted aryl-(C₁-C₈)alkyl, unsubstituted or substituted heteroaryl, unsubstituted or substituted heteroaryl-(C₁-C₈)alkyl, cyano, —COR^{a}, —CO₂R^{a}, —CONR^{a}R^{b}, —CONR^{a}NR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —NR^{a}NR^{a}R^{b}, —NR^{a}NR^{a}C(O)R^{b}, —NR^{a}NR^{a}C(O)NR^{a}R^{b}, —NR^{a}NR^{a}C(O)OR^{a}, —OR^{a}, —OC(O)R^{a}, —OC(O)NR^{a}R^{b};
wherein any (C₁-C₈)alkyl, (C₂-C₈)alkenyl, (C₂-C₈)alkynyl, cycloalkyl, cycloalkenyl, bicycloalkyl, heterocycloalkyl, aryl, or heteroaryl group is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, —OC(O)NR^{a}R^{b}, heterocycloalkyl, aryl, heteroaryl, aryl(C₁-C₄)alkyl, and heteroaryl(C₁-C₄)alkyl;
   wherein any aryl or heteroaryl moiety of said aryl, heteroaryl, aryl(C₁-C₄)alkyl, or heteroaryl(C₁-C₄)alkyl is optionally substituted by 1, 2 or 3 groups independently selected from the group consisting of halo, (C₁-C₆)alkyl, (C₃-C₈)cycloalkyl, (C₅-C₈)cycloalkenyl, (C₁-C₆)haloalkyl, cyano, —COR^{a}, —CO₂R^{a}, —CONR^{a}R^{b}, —SR^{a}, —SOR^{a}, —SO₂R^{a}, —SO₂NR^{a}R^{b}, nitro, —NR^{a}R^{b}, —NR^{a}C(O)R^{b}, —NR^{a}C(O)NR^{a}R^{b}, —NR^{a}C(O)OR^{a}, —NR^{a}SO₂R^{b}, —NR^{a}SO₂NR^{a}R^{b}, —OR^{a}, —OC(O)R^{a}, and —OC(O)NR^{a}R^{b};

R^{a} and R^{b} are each independently hydrogen, (C₁-C₈)alkyl, (C₂-C₈)alkenyl, (C₂-C₈)alkynyl, (C₃-C₈)cycloalkyl, (C₅-C₈)cycloalkenyl, (C₆-C₁₀)bicycloalkyl, heterocycloalkyl, aryl, heteroaryl, wherein said (C₁-C₈)alkyl, (C₂-C₈)alkenyl, (C₂-C₈)alkynyl, cycloalkyl, cycloalkenyl, bicycloalkyl, heterocycloalkyl, aryl or heteroaryl group is optionally substituted by 1, 2 or 3 groups independently selected from halo, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, —CON((C₁-C₄)alkyl)((C₁-C₄)alkyl), —SO₂(C₁-C₄)alkyl, —SO₂NH₂, —SO₂NH(C₁-C₄)alkyl, or —SO₂N((C₁-C₄)alkyl)((C₁-C₄)alkyl);

or R^{a} and R^{b} taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, and (C₁-C₄)alkoxy(C₁-C₄)alkyl, wherein said ring is optionally fused to a (C₃-C₈)cycloalkyl, heterocycloalkyl, aryl, or heteroaryl ring;

or R^{a} and R^{b} taken together with the nitrogen to which they are attached represent a 6- to 10-membered bridged bicyclic ring system optionally fused to a (C₃-C₈)cycloalkyl, heterocycloalkyl, aryl, or heteroaryl ring;

or a salt thereof.

In some embodiments, the EZH2 inhibitor is an EZH2 inhibitor described in Verma et al. ACS Med. Chem. Letters 3:1091-1096 (2012). In some embodiments, the EZH2 inhibitor is a compound of formula (IV):

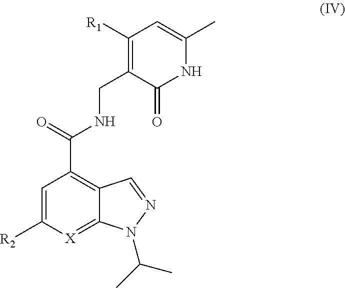

| | | | | EZH2 | H3K27me3 |
|---|---|---|---|---|---|
| Compound | R₁ | R₂ | X | Kᵢ^{app} (nM)^{a} | IC₅₀ (nM)^{b} |
| 1 | CH₃ | cyclopropyl | N | 149 ± 28 | 10,632 ± 4905 |
| 2 | CH₃ | cyclopropyl | CH | 74 ± 11 | 2,510 ± 960 |
| 3 (GSK926) | CH₃ | 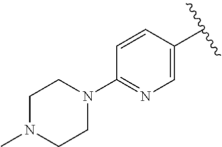 [SMILES: *c1ccc(N2CCN(C)CC2)nc1] | CH | 7.9 ± 3 | 324 ± 126 |
| 4 | n-propyl | 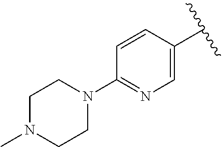 [SMILES: *c1ccc(N2CCN(C)CC2)nc1] | CH | 0.60 ± 0.05 | 79 ± 7 |
| 5 | CH₃ | 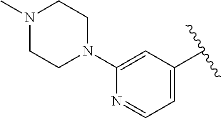 [SMILES: *c1ccnc(N2CCN(C)CC2)c1] | CH | 14 ± 5 | 1,995 ± 1384 |
| 6 (GSK343) | n-propyl | 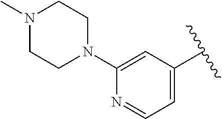 [SMILES: *c1ccnc(N2CCN(C)CC2)c1] | CH | 1.2 ± 0.2 | 174 ± 84 |

In some embodiments, the EZH2 inhibitor is a compound of Formula (Ig) or a pharmaceutically acceptable salt thereof:

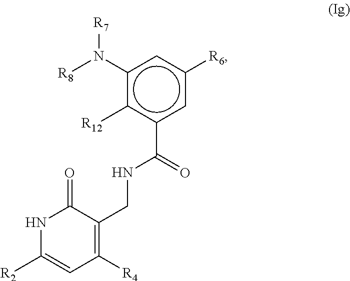

wherein R₂, R₄ and R₁₂ are each, independently C₁₋₆ alkyl;
R₆ is C₆-C₁₀ aryl or 5- or 6-membered heteroaryl, each of which is optionally substituted with one or more -Q₂-T₂, wherein Q₂ is a bond or C₁-C₃ alkyl linker optionally substituted with halo, cyano, hydroxyl or C₁-C₆ alkoxy, and T₂ is H, halo, cyano, —ORₐ, —NRₐR_{b}, —(NRₐR_{b}R_{c})⁺A⁻, —C(O)Rₐ, —C(O)ORₐ, —C(O)NRₐR_{b}, —NR_{b}C(O)Rₐ, —NR_{b}C(O)ORₐ, —S(O)₂Rₐ, —S(O)₂NRₐR_{b}, or R_{S2}, in which each of Rₐ, R_{b} and R_{c}, independently is H or R_{S3}, A⁻ is a pharmaceutically acceptable anion, each of R_{S2} and R_{S3}, independently, is C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S2}, R_{S3}, and the 4 to 12-membered heterocycloalkyl ring formed by Rₐ and R_{b}, is optionally substituted with one or more -Q₃-T₃, wherein Q₃ is a bond or C₁-C₃ alkyl linker each optionally substituted with halo, cyano, hydroxyl or C₁-C₆ alkoxy, and T₃ is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of R_{d} and Rₑ independently being H or C₁-C₆ alkyl, or -Q₃-T₃ is oxo; or any two neighboring -Q₂-T₂, together with the atoms to which they are attached form a 5- or 6-membered ring optionally containing 1-4 heteroatoms selected from N, O and S and optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;
R₇ is -Q₄-T₄, in which Q₄ is a bond, C₁-C₄ alkyl linker, or C₂-C₄ alkenyl linker, each linker optionally substituted with halo, cyano, hydroxyl or C₁-C₆ alkoxy, and T₄ is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR_{f}C(O)R_{g}, S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and each of R_{S4} and R_{S5} is optionally substituted with one or more -Q₅-T₅, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)_{q}R_{q} in which q is 0, 1, or 2 and R_{g} is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T₅ is H, halo, hydroxyl, or cyano; or -Q₅-T₅ is oxo; and
R₈ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, C₃-C₈ cycloalkyl, 4 to 12-membered heterocycloalkyl, amino, mono-C₁-C₆ alkylamino, or di-C₁-C₆ alkylamino, and R_{S6} is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring having 0 to 2 additional heteroatoms, and the 4 to 11-membered heterocycloalkyl ring formed by R₇ and R₈ is optionally substituted with one or more -Q₆-T₆, wherein Q₆ is a bond, C(O), C(O)NRₘ, NRₘC(O), S(O)₂, or C₁-C₃ alkyl linker, Rₘ being H or C₁-C₆ alkyl, and T₆ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₆ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl except when T₆ is H, halo, hydroxyl, or cyano; or -Q₆-T₆ is oxo.

In some embodiments, the EZH2 inhibitor is a compound is of Formula (II) or a pharmaceutically acceptable salt thereof:

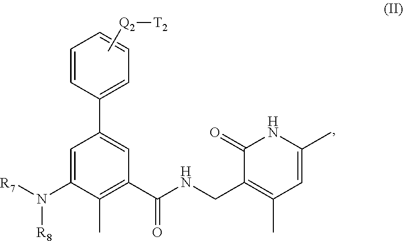

wherein Q₂ is a bond or methyl linker, T₂ is H, halo, —ORₐ, —NRₐR_{b}, —(NRₐR_{b}R_{c})⁺A⁻, or —S(O)₂NRₐR^{b}, R₇ is piperidinyl, tetrahydropyran, cyclopentyl, or cyclohexyl, each optionally substituted with one -Q₅-T₅ and R₈ is ethyl.

In some embodiments, the EZH2 inhibitor is a compound of Formula (IIa) or a pharmaceutically acceptable salt thereof:

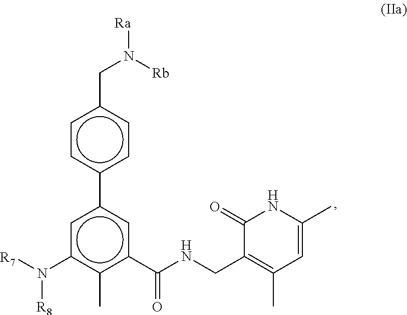

wherein each of Rₐ and R_{b}, independently is H or R_{S3}, R_{S3} being C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S3} and the 4 to 12-membered heterocycloalkyl ring formed by Rₐ and R_{b}, is optionally substituted with one or more -Q₃-T₃, wherein Q₃ is a bond or C₁-C₃ alkyl linker each optionally substituted with halo, cyano, hydroxyl or C₁-C₆ alkoxy, and T₃ is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of Rd and Rₑ independently being H or C₁-C₆ alkyl, or -Q₃-T₃ is oxo;
R₇ is -Q₄-T₄, in which Q₄ is a bond, C₁-C₄ alkyl linker, or C₂-C₄ alkenyl linker, each linker optionally substituted with halo, cyano, hydroxyl or C₁-C₆ alkoxy, and T₄ is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR_{f}C(O)R_{g}, —S(O)₂R_{f}, or R_{S4}, in which each of R_{f} and R_{g}, independently is H or R_{S5}, each of R_{S4} and R_{S5}, independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and each of R_{S4} and R_{S5} is optionally substituted with one or more -Q₅-T₅, wherein Q₅ is a bond, C(O), C(O)NRₖ, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, 5- or 6-membered heteroaryl, or S(O)_{q}R_{q} in which q is 0, 1, or 2 and R_{g} is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, and 5- or 6-membered heteroaryl except when T₅ is H, halo, hydroxyl, or cyano; or -Q₅-T₅ is oxo; provided that R₇ is not H; and
R₈ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, amino, mono-C₁-C₆ alkylamino, or di-C₁-C₆ alkylamino, and R_{S6} is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring which has 0 to 2 additional heteroatoms and is optionally substituted with one or more -Q₆-T₆, wherein Q₆ is a bond, C(O), C(O)NRₘ, NRₘC(O), S(O)₂, or C₁-C₃ alkyl linker, Rₘ being H or C₁-C₆ alkyl, and T₆ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 7-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, or 5- or 6-membered heteroaryl, and T₆ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈cycloalkyl, C₆-C₁₀ aryl, 4 to 7-membered heterocycloalkyl, and 5- or 6-membered heteroaryl except when T₆ is H, halo, hydroxyl, or cyano; or -Q₆-T₆ is OXO.

In some embodiments, the EZH2 inhibitor is wherein the EZH2 inhibitor is Compound B:

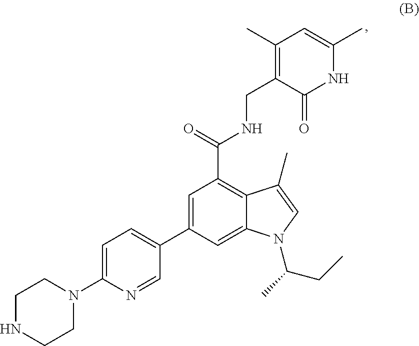 [SMILES: CC[C@H](C)n1cc(C)c2c(C(=O)NCc3c(C)cc(C)[nH]c3=O)cc(-c3ccc(N4CCNCC4)nc3)cc21]

or a pharmaceutically acceptable salt thereof.

In some embodiments, the EZH2 inhibitor is EPZ-6438. In some embodiments, the EZH2 inhibitor is EPZ-6438 as described in Knutson et al. PNAS 110(9):7922-7927 (2013), which is hereby incorporated by reference in its entirety. In some embodiments, the EZH2 inhibitor is CAS #: 1403254-99-8 or a pharmaceutically acceptable salt thereof. In some embodiments, the EZH2 inhibitor is N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide or a pharmaceutically acceptable salt thereof. In some embodiments, the EZH2 inhibitor is Compound Z:

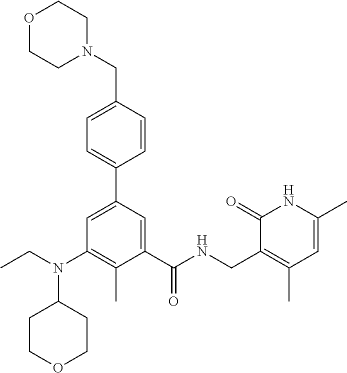 [SMILES: CCN(c1cc(-c2ccc(CN3CCOCC3)cc2)cc(C(=O)NCc2c(C)cc(C)[nH]c2=O)c1C)C1CCOCC1]

or a pharmaceutically acceptable salt thereof.

Provided herein are also HDAC inhibitors useful in the methods described herein. Histone deacetylases (HDAC) are a class of enzymes that remove acetyl groups (O═C—CH3) from an ε-N-acetyl lysine amino acid on a histone. HDAC are classified in four classes depending on sequence identity and domain organization. Class 1 HDACs include HDAC1, HDAC2, HDAC3, and HDAC8. Class HA HDACs include HDAC4, HDAC5, HDAC7, and HDAC9. Class IIB HDACs include HDAC6 and HDAC7. Class III HDACs include sirtuins (SIRT1-7). Class IV HDACs include HDAC11. In some embodiments, the HDAC inhibitor is Trichostatin A (TSA) and/or suberoylanilide hydroxamic acid (SAHA) which inhibits Class I and Class II HDACs. In some embodiments, the HDAC inhibitor is MS-275 which inhibits HDACs 1, 2, 3, and 8. In some embodiments, the HDAC inhibitor is Scriptaid. In some embodiments, the HDAC inhibitor is a HDAC Class 1 inhibitor. In some embodiments, the HDAC inhibitor inhibits the deacetylase activity of one, two, three, or four HDACs of Class I. In some embodiments, the HDAC inhibitor inhibits the deacetylase activity of HDAC1, 2, and 3 and not HDAC8. In some embodiments, the HDAC inhibitor inhibits the deacetylase activity of HDAC3 and not HDAC 1, 2, and/or 8. In some embodiments, the HDAC inhibitor inhibits the deacetylase activity of HDAC2 and not HDAC 1, 3, and/or 8. In some embodiments, the HDAC inhibitor inhibits the deacetylase activity of HDAC1 and 2 and not HDAC 3 and/or 8. In some embodiments, the HDAC inhibitor inhibits histone H3 K27 deacetylation (increases H3 K27 acetylation). In some embodiments, the HDAC inhibitor results in an increase histone H3 K27 tri-methylation.

In some embodiments, the HDAC inhibitor is G946 or a pharmaceutically acceptable salt thereof, wherein G946 is

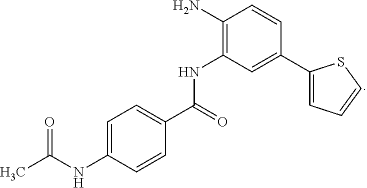 [SMILES: CC(=O)Nc1ccc(C(=O)Nc2cc(-c3cccs3)ccc2N)cc1]

In some embodiments, the HDAC inhibitor is G877 or a pharmaceutically acceptable salt thereof, wherein G877 is

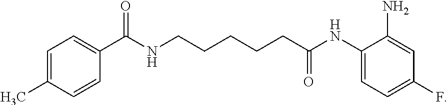 [SMILES: Cc1ccc(C(=O)NCCCCCC(=O)Nc2ccc(F)cc2N)cc1]

In some embodiments, the HDAC inhibitor is one or more of (I) Hydroxamic acids (such as trichostatin A (TSA), oxamflatin, and hydroxamic acid-based hybrid polar compounds such as suberoylanilide hydroxamic acid (SAHA) and pyroxamide; (II) Cyclic tetrapeptides with the epoxyketone-containing amino acid (2S,9S)-2-amino-8-oxo-9,10-epoxydecanoyl (Aoe) (such as trapoxin A and B, Cyl-1 and Cyl-2, HC-toxin, WF-3161, chlamydocin); (III) Cyclic tetrapeptides without Aoe (such as apicidin and the depsipeptide FR-901228); and/or (IV) Short-chain and aromatic fatty acids (such as butyrate, 4-phenybutyrate, and valproic acid); (V) Benzamides (such as MS-275). N some embodiments, the HDAC inhibitor is one or more of Givinostat (ITF2357), LAQ 824, Belinostat (PXD 101), PCI 24781, Romidepsin (FK 228), Entinostat (MS275-SNDX275), Mocetinostat (MGCD0103), YM753, valproic acid (VPA), vironostat (SAHA). Tacedinalien (CI 994). Examples of HDAC inhibitors include, but are not limited to, U.S. Pat. No. 7,399,787, US2009023786, US2009/270351, US2009/076101, US2009/239849, US2009/069391, US2009/215813, WO2009/045385, WO2009/020589, WO2009/005638, WO2009/002495, US2009/012075, US2009/118291, EP2091525, WO2009/014941, US2009/209596, WO2009/003625, WO2009/117831, and/or WO2009/126877, which are incorporated by reference in their entirety.

Provided here are also EGFR antagonists useful in the methods described herein. EGFR is meant the receptor tyrosine kinase polypeptide Epidermal Growth Factor Receptor which is described in Ullrich et al., Nature (1984) 309:418425, alternatively referred to as Her-1 and the c-erbB gene product, as well as variants thereof such as EGFRvIII. Variants of EGFR also include deletional, substitutional and insertional variants, for example those described in Lynch et al. (NEJM 2004, 350:2129), Paez et al. (Science 2004, 304:1497), Pao et al. (PNAS 2004, 101:13306). In some embodiment, the EGFR is wild-type EGFR, which generally refers to a polypeptide comprising the amino acid sequence of a naturally occurring EGFR protein. In some embodiments, the EGFR antagonists are an antibody, binding polypeptide, binding small molecule, and/or polynucleotide.

Exemplary EGFR antagonists (anti-EGFR antibodies) include antibodies such as humanized monoclonal antibody known as nimotuzumab (YM Biosciences), fully human ABX-EGF (panitumumab, Abgenix Inc.) as well as fully human antibodies known as E1.1, E2.4, E2.5, E6.2, E6.4, E2.11, E6. 3 and E7.6. 3 and described in U.S. Pat. No. 6,235,883; MDX-447 (Medarex Inc). Pertuzumab (2C4) is a humanized antibody that binds directly to HER2 but interferes with HER2-EGFR dimerization thereby inhibiting EGFR signaling. Other examples of antibodies which bind to EGFR include GA201 (RG7160; Roche Glycart AG), MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). In some embodiments, the anti-EGFR antibody is cetuximab. In some embodiments, the anti-EGFR antibody is panitumumab. In some embodiments, the anti-EGFR antibody is zalutumumab, nimotuzumab, and/or matuzumab.

Anti-EGFR antibodies that are useful in the methods include any antibody that binds with sufficient affinity and specificity to EGFR and can reduce or inhibit EGFR activity. The antibody selected will normally have a sufficiently strong binding affinity for EGFR, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g., RIA's), for example. Preferably, the anti-EGFR antibody of the invention can be used as a therapeutic agent in targeting and interfering with diseases or conditions wherein EGFR/EGFR ligand activity is involved. Also, the antibody may be subjected to other biological activity assays, e.g., in order to evaluate its effectiveness as a therapeutic. Such assays are known in the art and depend on the target antigen and intended use for the antibody. In some embodiments, a EGFR arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the EGFR-expressing cell. Bispecific antibodies may also be used to localize cytotoxic agents to cells which express EGFR. These antibodies possess an EGFR-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

Exemplary EGFR antagonists also include small molecules such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, WO9935146, WOO 0132651, U.S. Pat. No. 6,344,455, U.S. Pat. No. 5,760,041, U.S. Pat. No. 6,002,008, and/or U.S. Pat. No. 5,747,498. Particular small molecule EGFR antagonists include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa® (ZD1839, gefitinib, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); lapatinib (Tykerb, GlaxoSmithKline); ZD6474 (Zactima, AstraZeneca); CUDC-101 (Curis); canertinib (CI-1033); AEE788 (6-[4-[(4-ethyl-1-piperazinyl)methyl]phenyl]-N-[(1R)-1-phenylethyl]-7H-pyrrolo[2,3-d]pyrimidin-4-amine, WO2003013541, Novartis) and PKI166 4-[4-[[(1R)-1-phenylethyl]amino]-7H-pyrrolo[2,3-d]pyrimidin-6-yl]-phenol, WO9702266 Novartis). In some embodiments, the EGFR antagonist is N-(3-ethynylphenyl)-6,7-bis(2-methoxyethoxy)-4-quinazolinamine and/or a pharmaceutical acceptable salt thereof (e.g., N-(3-ethynylphenyl)-6,7-bis(2-methoxyethoxy)-4-quinazolinamine-HCl). In some embodiments, the EGFR antagonist is gefitinib and/or a pharmaceutical acceptable salt thereof. In some embodiments, the EGFR antagonist is lapatinib and/or a pharmaceutical acceptable salt thereof. In some embodiments, the EGFR antagonist is gefitinib and/or erlotinib.

In some embodiments, the EGFR antagonist may be a specific inhibitor for EGFR. In some embodiments, the inhibitor may be a dual inhibitor or pan inhibitor wherein the EGFR antagonist inhibits EGFR and one or more other target polypeptides.

Provided here are also taxanes useful in the methods described herein. Taxanes are diterpenes which may bind to tubulin, promoting microtubule assembly and stabilization and/or prevent microtubule depolymerization. Taxanes included herein taxoid 10-deacetylbaccatin III and/or derivatives thereof. Examples to taxanes include, but are not limited to, paclitaxel (i.e., taxol, CAS #33069-62-4), docetaxel (i.e., taxotere, CAS #114977-28-5), larotaxel, cabazitaxel, milataxel, tesetaxel, and/or orataxel. In some embodiments, the taxane is paclitaxel. In some embodiments, the taxane is docetaxel. In some embodiments, the taxane is formulated in Cremophor (e.g., Taxol®) to Tween such as polysorbate 80 (e.g., Taxotere®). In some embodiments, the taxane is liposome encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of Cremophor and/or Tween-such as Tocosol Paclitaxel). In some embodiments, the taxane is an albumin-coated nanoparticle (e.g., Abraxane and/or ABI-008). In some embodiments, the taxane is Taxol®.

A. Antibodies

Provided herein isolated antibodies that bind to a polypeptide of interest, such as a chromatin modifier and/or EGFR for use in the methods described herein. In any of the above embodiments, an antibody is humanized. Further, the antibody according to any of the above embodiments is a monoclonal antibody, including a chimeric, humanized or human antibody. In one embodiment, the antibody is an antibody fragment, e.g., a Fv, Fab, Fab′, scFv, diabody, or F(ab′)₂ fragment. In another embodiment, the antibody is a full length antibody, e.g., an “intact IgG1” antibody or other antibody class or isotype as defined herein.

In a further aspect, an antibody according to any of the above embodiments may incorporate any of the features, singly or in combination, as described in Sections below:

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g., 10⁻⁸ M or less, e.g., from 10⁻⁸ M to 10⁻¹³ M, e.g., from 10⁻⁹ M to 10⁻¹³ M). In one embodiment, Kd is measured by a radiolabeled antigen binding assay (RIA). In one embodiment, the RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO m spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthtün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity-determining region (SDR) grafting); Padlan, Mol. Immunol. 28:489-498 (1991) (describing “resurfacing”); Dall' Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Hist. & Histopath., 20(3):927-937 (2005) and Vollmers and Brandlein, Methods Find Exp. Clin. Pharmacol., 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods Mol. Biol. 178:1-37 (O'Brien et al., ed., Human Press, Totowa, N. J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods Mol. Biol. 248:161-175 (Lo, ed., Human Press, Totowa, N. J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is a polypeptide of interest, such as a chromatin modifier and/or EGFR and the other is for any other antigen. In certain embodiments, bispecific antibodies may bind to two different epitopes of a polypeptide of interest, such as chromatin modifier and/or EGFR. Bispecific antibodies may also be used to localize cytotoxic agents to cells which express a polypeptide of interest, such as chromatin modifier and/or EGFR. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to a polypeptide of interest, such as chromatin modifier and/or EGFR as well as another, different antigen (see, US 2008/0069820, for example).

7. Antibody Variants

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).
b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII) only, whereas monocytes express Fc(RI), Fc(RII) and Fc(RIII). FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

B. Immunoconjugates

Further provided herein are immunoconjugates comprising antibodies which bind a polypeptide of interest such as a chromatin modifier antibody or EGFR, conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes for use in the methods described herein.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC 1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

C. Binding Polypeptides

Binding polypeptides are polypeptides that bind, preferably specifically, to a polypeptide of interest such as a chromatin modifier and/or EGFR are also provided for use in the methods described herein. In some embodiments, the binding polypeptides are chromatin modifier antagonists and/or a targeted therapy (e.g., EGFR antagonists). Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, e.g., a chromatin modifier or EGFR, as described herein. Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

D. Binding Small Molecules

Provided herein are binding small molecules for use as a small molecule antagonist of a chromatin modifier, a targeted therapy (e.g., small molecule EGFR antagonist), and/or chemotherapy (e.g., taxane) for use in the methods described above.

Binding small molecules are preferably organic molecules other than binding polypeptides or antibodies as defined herein that bind, preferably specifically, to a chromatin modifier and/or EGFR as described herein. Binding organic small molecules may be identified and chemically synthesized using known methodology (see, e.g., PCT Publication Nos. WO00/00823 and WO00/39585). Binding organic small molecules are usually less than about 2000 daltons in size, alternatively less than about 1500, 750, 500, 250 or 200 daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide of interest are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

E. Antagonist Polynucleotides

Provided herein are also polynucleotide antagonists for use in the methods described herein. The polynucleotide may be an antisense nucleic acid and/or a ribozyme. The antisense nucleic acids comprise a sequence complementary to at least a portion of an RNA transcript of a gene of interest, such as a chromatin modifier gene described herein and/or EGFR gene. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double stranded antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the gene, could be used in an antisense approach to inhibit translation of endogenous mRNA. Polynucleotides complementary to the 5′ untranslated region of the mRNA should include the complement of the AUG start codon. Antisense polynucleotides complementary to mRNA coding regions are less efficient inhibitors of translation but could be used in accordance with the invention. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

F. Antibody and Binding Polypeptide Variants

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., antigen-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody and/or binding polypeptide of interest and the products screened for a desired activity, e.g., retained/improved antigen binding, decreased immunogenicity, or improved ADCC or CDC.

TABLE 1

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

G. Antibody and Binding Polypeptide Derivatives

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody and/or binding polypeptide-nonproteinaceous moiety are killed.

### IV. Methods of Screening and/or Identifying Antagonists of a Chromatin Modifier with Desired Function

Additional antagonists of a polypeptide of interest, such as a chromatin modifier and/or EGFR for use in the methods described herein, including antibodies, binding polypeptides, and/or small molecules have been described above. Additional antagonists of such as anti-chromatin modifier antibodies, binding polypeptides, and/or binding small molecules provided herein may be identified, screened for, or characterized for their physical/chemical properties and/or biological activities by various assays known in the art.

In certain embodiments, a computer system comprising a memory comprising atomic coordinates of a chromatin modifier polypeptide are useful as models for rationally identifying compounds that a ligand binding site of a chromatin modifier. Such compounds may be designed either de novo, or by modification of a known compound, for example. In other cases, binding compounds may be identified by testing known compounds to determine if the “dock” with a molecular model of a chromatin modifier. Such docking methods are generally well known in the art.

The chromatin modifier crystal structure data can be used in conjunction with computer-modeling techniques to develop models of binding of various chromatin modifier-binding compounds by analysis of the crystal structure data. The site models characterize the three-dimensional topography of site surface, as well as factors including van der Waals contacts, electrostatic interactions, and hydrogen-bonding opportunities. Computer simulation techniques are then used to map interaction positions for functional groups including but not limited to protons, hydroxyl groups, amine groups, divalent cations, aromatic and aliphatic functional groups, amide groups, alcohol groups, etc. that are designed to interact with the model site. These groups may be designed into a pharmacophore or candidate compound with the expectation that the candidate compound will specifically bind to the site. Pharmacophore design thus involves a consideration of the ability of the candidate compounds falling within the pharmacophore to interact with a site through any or all of the available types of chemical interactions, including hydrogen bonding, van der Waals, electrostatic, and covalent interactions, although in general, pharmacophores interact with a site through non-covalent mechanisms.

The ability of a pharmacophore or candidate compound to bind to a chromatin modifier polypeptide can be analyzed in addition to actual synthesis using computer modeling techniques. Only those candidates that are indicated by computer modeling to bind the target (e.g., a chromatin modifier polypeptide binding site) with sufficient binding energy (in one example, binding energy corresponding to a dissociation constant with the target on the order of 10⁻² M or tighter) may be synthesized and tested for their ability to bind to a chromatin modifier polypeptide and to inhibit a chromatin modifier, if applicable, enzymatic function using enzyme assays known to those of skill in the art and/or as described herein. The computational evaluation step thus avoids the unnecessary synthesis of compounds that are unlikely to bind a chromatin modifier polypeptide with adequate affinity.

A chromatin modifier pharmacophore or candidate compound may be computationally evaluated and designed by means of a series of steps in which chemical entities or fragments are screened and selected for their ability to associate with individual binding target sites on a chromatin modifier polypeptide. One skilled in the art may use one of several methods to screen chemical entities or fragments for their ability to associate with a chromatin modifier polypeptide, and more particularly with target sites on a chromatin modifier polypeptide. The process may begin by visual inspection of, for example a target site on a computer screen, based on the chromatin modifier polypeptide coordinates, or a subset of those coordinates known in the art.

To select for an antagonist which induces cancer cell death, loss of membrane integrity as indicated by, e.g., propidium iodide (PI), trypan blue or 7AAD uptake may be assessed relative to a reference. A PI uptake assay can be performed in the absence of complement and immune effector cells. A tumor cells are incubated with medium alone or medium containing the appropriate combination therapy. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those antagonists that induce statistically significant levels of cell death compared to media alone and/or monotherapy as determined by PI uptake may be selected as cell death-inducing antibodies, binding polypeptides or binding small molecules.

In some embodiments of any of the methods of screening and/or identifying, the candidate antagonist of a chromatin modifier is an antibody, binding polypeptide, binding small molecule, or polynucleotide. In some embodiments, the antagonist of a chromatin modifier is an antibody. In some embodiments, the antagonist of a chromatin modifier is a small molecule.

### V. Pharmaceutical Formulations

Pharmaceutical formulations of a modulator of a chromatin modifier (e.g., an antagonist of a chromatin modifier) and/or a cancer therapy agent (e.g., targeted therapy, chemotherapy, and/or radiotherapy) as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, the antagonist of a chromatin modifier and/or targeted therapy is a binding small molecule, an antibody, binding polypeptide, and/or polynucleotide. In some embodiments, the cancer therapy agent is EGFR antagonist. In some embodiments, the cancer therapy agent is a chemotherapy. In some embodiments, the chemotherapy is a taxane. In some embodiments, the taxane is paclitaxel. In some embodiments, the taxane is docetaxel. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist of a chromatin modifier and/or cancer therapy agent (e.g., targeted therapy and/or chemotherapy) which matrices are in the form of shaped articles, e.g., films, or microcapsules.

The formulations to be used for in vivo administration are generally sterile. Sterility may be readily accomplished, e.g., by filtration through sterile filtration membranes.

### VI. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a modulator of a chromatin modifier (e.g., an antagonist of a chromatin modifier) described herein. The label or package insert indicates that the composition is used for treating the condition of choice. Moreover, the article of manufacture may comprise (a) a first container with a composition contained therein, wherein the composition comprises a modulator of a chromatin modifier (e.g., an antagonist of a chromatin modifier) and (b) a second container with a composition contained therein, wherein the composition comprises a cancer therapy agent (e.g., a targeted therapy and/or chemotherapy).

In some embodiments, the article of manufacture comprises a container, a label on said container, and a composition contained within said container; wherein the composition includes one or more reagents (e.g., primary antibodies that bind to one or more biomarkers or probes and/or primers to one or more of the biomarkers described herein), the label on the container indicating that the composition can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers described herein.

In some embodiments of any of the article of manufacture, the antagonist of a chromatin modifier and/or the cancer therapy agent (e.g., a targeted therapy) is an antibody, binding polypeptide, binding small molecule, or polynucleotide. In some embodiments, the cancer therapy agent is a taxane. In some embodiments, the taxane is paclitaxel. In some embodiments, the cancer therapy agent is an EGFR antagonist. In some embodiments, the antagonist of a chromatin modifier and/or EGFR antagonist is a small molecule. In some embodiments, the EGFR small molecule antagonist is erlotinib and/or gefitinib. In some embodiments, the antagonist of a chromatin modifier and/or EGFR antagonist is an antibody. In some embodiments, the antibody is a monoclonal antibody. In some embodiments, the antibody is a human, humanized, or chimeric antibody. In some embodiments, the antibody is an antibody fragment and the antibody fragment binds a chromatin modifier and/or inhibitor.

The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

It is understood that any of the above articles of manufacture may include an immunoconjugate described herein in place of or in addition to a modulator of a chromatin modifier (e.g., an antagonist of a chromatin modifier) and a cancer therapy agent (e.g., an EGFR antagonist or taxane (e.g., paclitaxel)).

### EXAMPLES

The following are examples of methods and compositions of the invention. It is understood that various other embodiments may be practiced, given the general description provided above.

### Example 1

To identify gene products involved in the chromatin alterations which result in the survival of cancer cells during standard of care or targeted drug treatment of cancer cells, histone mass spectrometry and a siRNA screen were initiated. In particular, histone mass spectrometry of parental cells and drug tolerant populations (DTPs) cells were used to identify histone tail modifications altered in the DTPs compared to the parental cell lines. Further, an siRNA library of approximately 300 chromatin modifiers was screened which included histone demethylases, methyltransferases histone acetyltransferases, histone deacetylases, bromodomain containing proteins, ubiquinase and deubiquinase enzymes, as well as histone chaperones (4 different siRNA sequences for each gene).

### Materials and Methods

Cell Culture

All cells are maintained in RPMI media (high glucose) supplemented with 5% Fetal Bovine Serum (FBS) and L-glutamine under 5% CO₂ at 37° C.

Cell Survival Assays

3×10⁴ cells were plated in each well of a 12-well cluster dish. 24 hours after plating, media was removed and replaced with media containing drugs. Fresh media was replaced every 2 days until untreated cells reached confluence. Media was then removed, cells were washed with Phosphate Buffered Saline (PBS), and then fixed for 15 min with 4% formaldehyde in PBS. Cells were then washed with PBS and stained with the fluorescent nucleic acid stain, Syto60 (1 nM in PBS; Molecular Probes) for 15 min. Dye was removed, cell monolayers were washed with PBS, and fluorescence quantitation was carried out at 700 nm with an Odyssey Infrared Imager (Li-Cor Biosciences).

Generation of Drug-Tolerant Persisters (DTPs)

Drug-sensitive cells (e.g., PC9 and H1299) were treated with relevant drug as described herein at concentrations exceeding 100 times the established IC₅₀ values, for three rounds, with each treatment lasting 72 hours. Viable cells remaining attached on the dish at the end of the third round of relevant drug treatment were considered to be DTPs, and were collected for analysis.

Cell Harvesting and Protein Analysis

Cell lysates were prepared in Laemmli sample buffer and analyzed by immunoblotting as described previously. Cell lysates were analyzed using commercial antibodies against modifications on H3 (Active Motif and Cell Signaling Technologies).

siRNA Library Creation

Through database searching (e.g., Pfam) for bromo, chromo and PHD domains and ontology keywords (e.g. acetyltransferase, deacetylase, methyltransferase, demethylase) as well as literature search, a siRNA library targeting 300 genes in the epigenetics space was assembled (see Table 2). Four single siRNAs targeting distinct regions on the target mRNA were utilized as unmodified siGENOME siRNAs.

siRNA Screening Methods

Cells (e.g., PC9 and H1299) were reverse transfected in black 96 well clear bottom plates (Coming, catalog #3603) at 1000 cell per well using 0.0625 ul of DharmaFECT 1 transfection lipid (Dharmacon, catalog #T-2001) and single siRNA (Dharmacon siGENOME) at 12.5 nM final concentration. Cells (e.g., PC9 and H1299) were subsequently transfected for 48-72 hours before replacing the transfection media by either 1 uM relevant drug treatment in media or media alone. After 72 hours of incubation the media +/− drug was then replaced with fresh media to enable recovery of the drug tolerant persisters (DTPs) that survived after the relevant drug treatment (recovery phase). After 3 days recovery phase, final cell viability was measured using CyQUANT Direct cell proliferation assay (Molecular Probes) according to the manufacturer protocol. CyQUANT fluorescent signal was detected using a GE IN Cell Analyzer 2000 (4× objective) and quantified as number of cell per well using an image analysis algorithm developed using GE Developer Tollbox 1.9.1. Screening data were subsequently processed in Microsoft Excel. The entire Epi300 siRNA screen was run on each cell line twice in completely independent conditions.

3-Deazaneplanocin A (DZNep) Cell Treatment

PC9 cells were seeded in black 96 well clear bottom plates (Coming, catalog #3603) at 1000 cells per well before to be treated with various concentration of 3-deazaneplanocin A (DZNep) from 40 down to 0.625 μM. After 48 hours of DZNep treatment the media was replaced with fresh media alone or in presence of 1 μM erlotinib. After 72 hours of incubation the media +/− erlotinib was then replaced with fresh media to enable recovery of the drug tolerant persisters (DTPs) that survived after the drug treatment. After 3 days recovery phase, final cell viability was measured using CyQUANT Direct cell proliferation assay (Molecular Probes) according to the manufacturer protocol. CyQUANT fluorescent signal was detected using a GE IN Cell Analyzer 2000 (4× objective) and quantified as number of cell per well using an image analysis algorithm developed using GE Developer Tollbox 1.9.1. Screening data were subsequently processed in Microsoft Excel. The entire Epi300 siRNA screen was run on each cell line twice in completely independent conditions.

Screen Quality Assessment

The quality of screens were assessed using Z-factors calculated based on the difference between the media and relevant drug treatment conditions for the non-targeting control (NTC) as well as between the non-targeting control and the positive control (HDAC3 siRNA single 3) (Dharmacon, catalog #D-003496-03) in the relevant drug treatment condition. For screens the Z-factor values comparing these conditions was between 0.5 and 1.

Mass Spectrometry Sample Preparation

Samples with 10 million cells were lysed and histones were isolated from cell lysates using the Active Motif Histone Purification Kit (world wide web activemotif.com/catalog/171.html). Protein quantitation post-isolation was performed using the Qubit fluorescence platform (Invitrogen). The target yield was at least 20 g or greater of purified histone per 5 million cells. The samples were then derivatized and binary comparisons using d0/d10 propionic anhydride and trypsin digestion was conducted. Specifically, 5 g aliquot of each sample was derivatized with d0 propionic anhydride to block lysine and mono-methylated lysine residues. The control sample utilized 15 g. Samples were digested with trypsin. Control sample were re-derivatized (on exposed peptide N-termini) with d0 propionic anhydride. Test samples were re-derivatized (on exposed N-termini) with d10 propionic anhydride. Each test sample was independently pooled 1:1 with control sample. Then the samples were subjected to multi-enzyme digestion. A suite of three enzymes per sample was employed to generate large peptides around the PTM sites to be characterized, and concomitant overlapping sequence coverage around all sites.

Mass Spectrometry

Peptide digests were analyzed by nano LC/MS/MS in data-dependent mode on a LTQ Orbitrap Velos tandem mass spectrometer. Data was acquired using CID, HCD and ETD fragmentation regimes. Upon data acquisition, database searching using Mascot (Matrix Science) was used to determine acetylation, methylation, dimethlyation, trimethylation, phosphorylation and ubiquitination. Manual data analysis including de novo sequencing was used to confirm putative in-silico assignments and interrogate raw data for modified peptides not matched in Mascot. Accurate mass full scan LC/MS data was integrated to determine relative abundance of modified peptides between samples. Trypsin-digested propionylated samples were quantitated within each LC/MS run by comparing d0/d5 pairs (according to the work of Garcia et al., JPR, 8, 5367-5374 (2009)). Alternate enzyme samples were quantitated label-free between LC/MS runs.

Xenograft Tumor Studies

PC9 cells were cultured in growth media (RPMI 1640, 10% heat-inactivated fetal calf serum, 2 mM L-glutamine) to 80% confluency and then trypsinized, washed once with PBS, and resuspended in either Hank's Balanced Salt Solution (HBSS) or a 1:1 mixture of HBSS with matrigel [growth factor reduced; catalog #356231 (BD Biosciences, West Grove, Pa.)] to a final concentration of 5×10⁷ cells/ml. Each xenograft tumor model was established using 5×10⁶ cells (100 μL) inoculated subcutaneously (s.c.) in the rear right flank of immunocompromised mice. PC-9 and PC-9-GFP cells were implanted in HBSS with matrigel in nude (nu/nu) mice (Charles River Laboratories, Hollister, Calif.). When tumor volumes reach approximately 100-200 mm³, mice were separated into groups of animals with similarly sized tumors, and treatment was initiated the day after grouping. Mice were dosed for 5 days a week (QD) oral gavage (PO) with erlotinib (50 mg/kg in 7.5% Captisol for first 4 doses then lowered to 35 mg/kg in 7.5% Captisol) and/or TSA (0.5 mg/kg) with appropriate vehicle control.

### Results

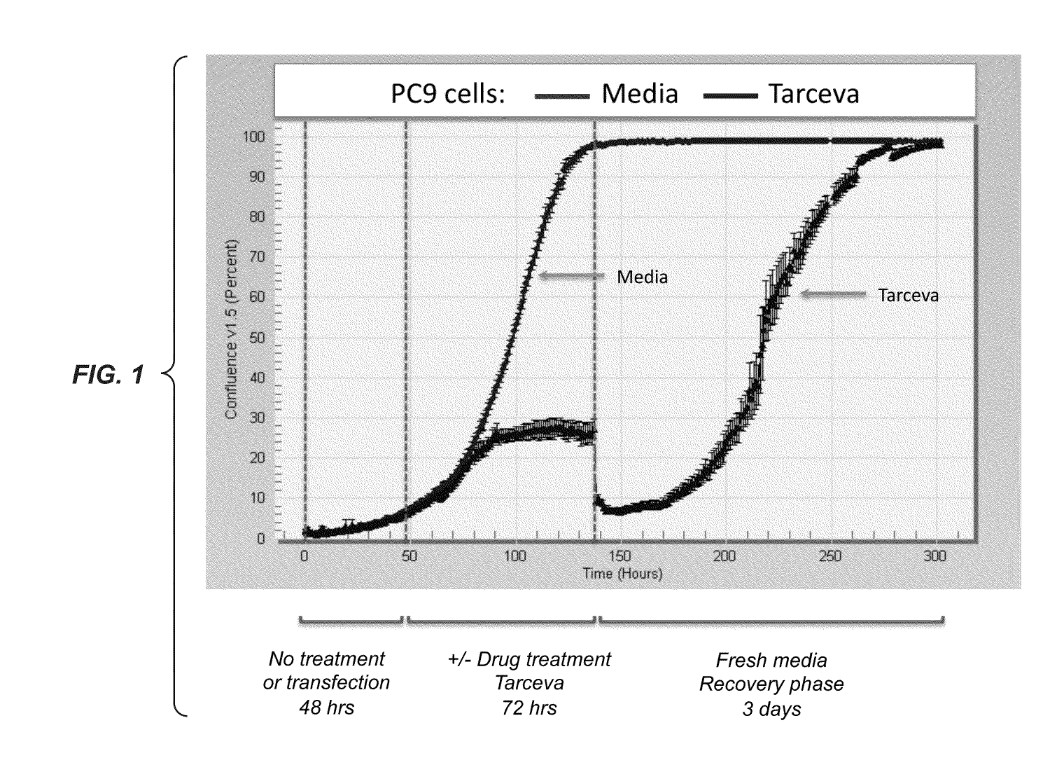

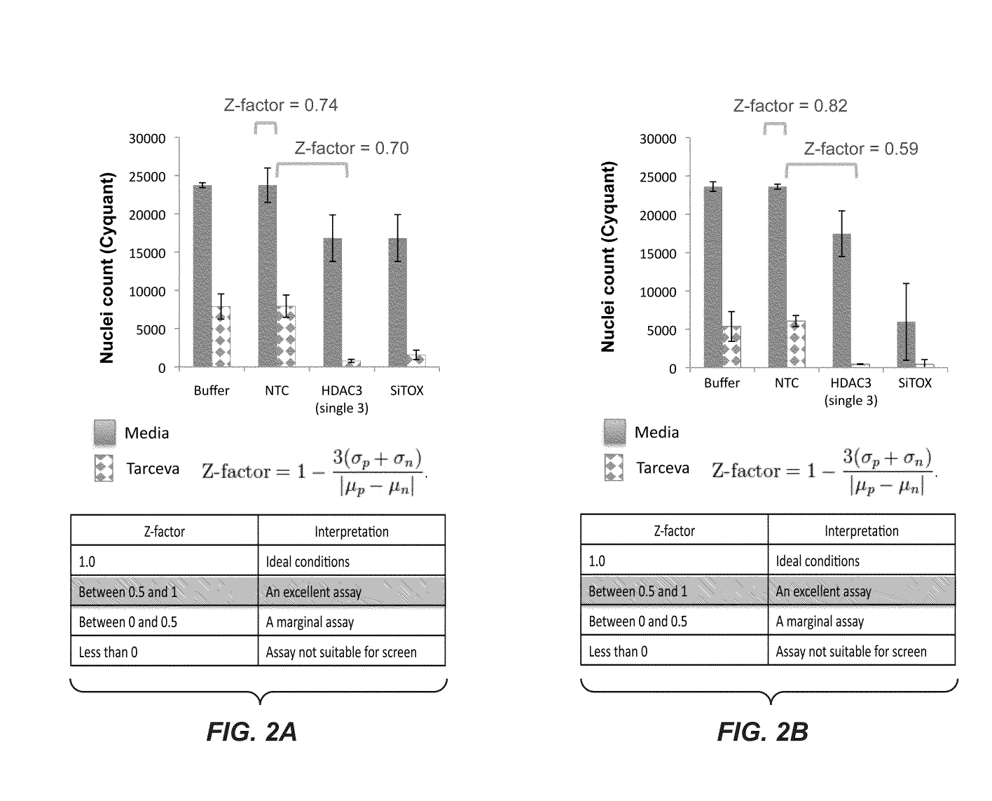

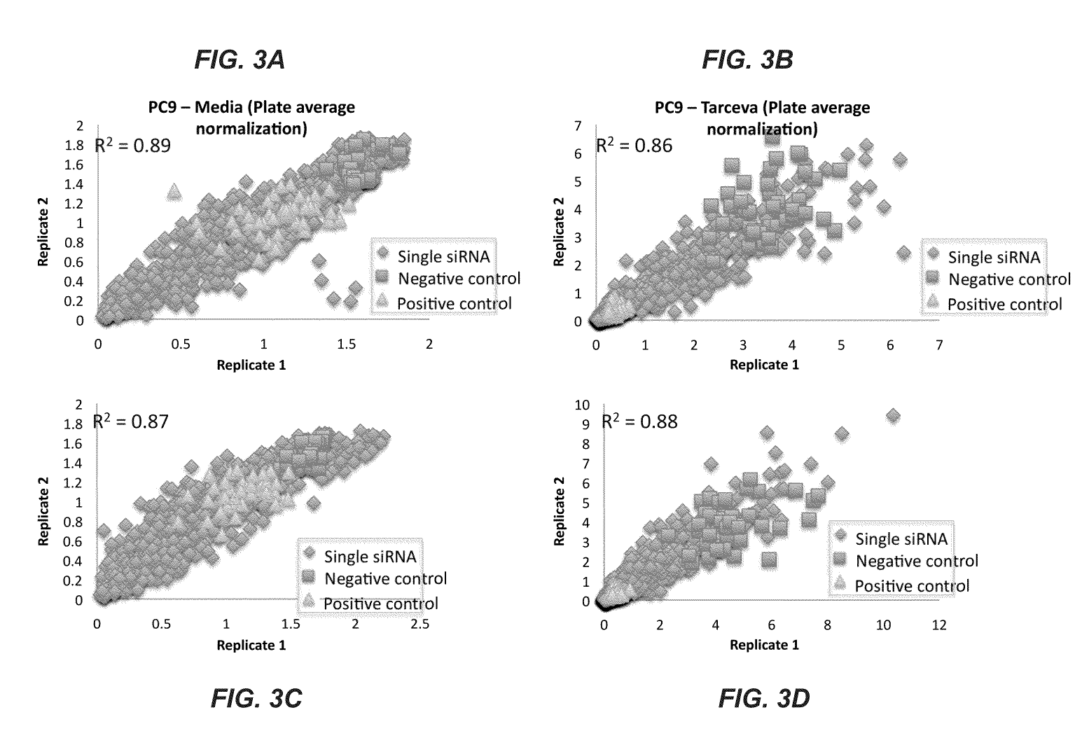

A siRNA screen was developed and implemented using the human non-small-cell lung cancer cell line PC9 in the context of drug tolerant persisters (DTPs) (FIG. 1). PC9 DTP cells were prepared and screened as described above. The cell number per well was normalized by the average cell number per well per plate for every condition (1200 single siRNAs) in both media and relevant drug, erlotinib, treatment. The quality of the screen was assessed using Z-factors calculated based on the difference between the media and erlotinib treatment conditions for the non-targeting control (NTC), as well as between the non-targeting control and the positive control (HDAC3 siRNA single 3)(Dharmacon, catalog #D-003496-03) in the erlotinib treatment condition (FIG. 2). For the screen, the Z-factor values comparing these conditions were between 0.5 and 1. Correlation between duplicate well ran across different plates was calculated (FIG. 3). A strong correlation between replicate plates (R²>0.8) was observed.

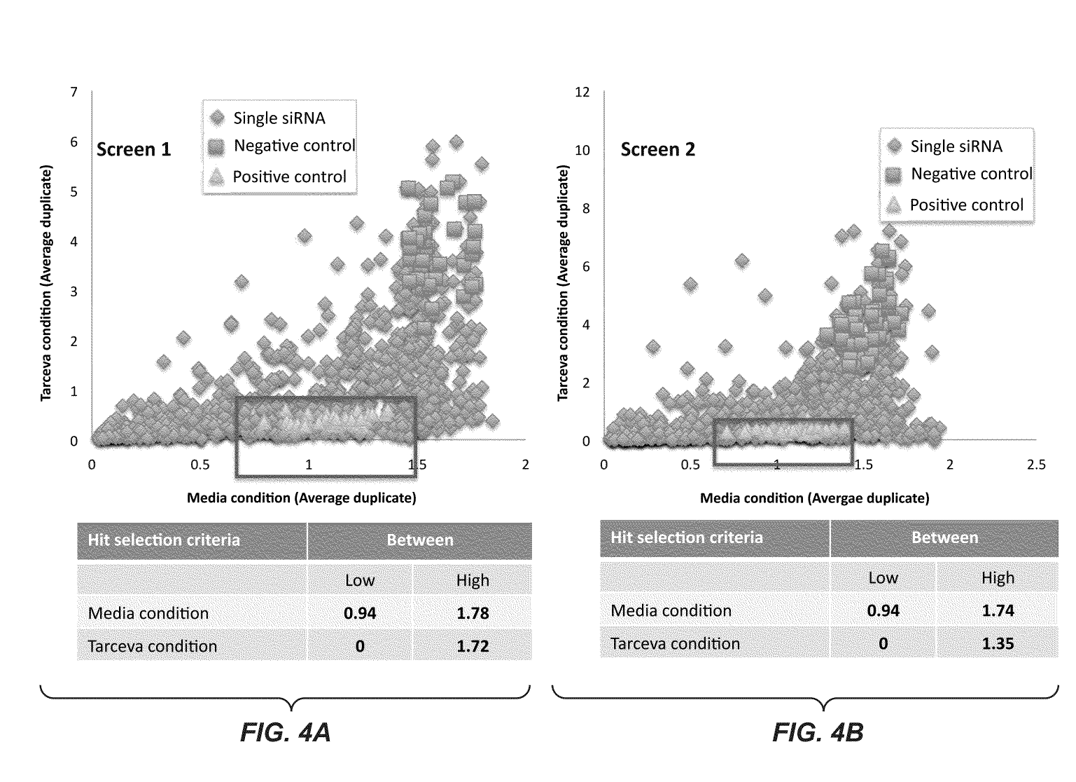

Positive hits were defined based on the effect of the specific gene knockdown in the media condition versus the erlotinib condition. Cut-off values were determine based on the variation of the positive control (HDAC3 siRNA single 3) and the negative control (non-targeting control) in order to extract positive hits with minimum effect in the media condition and a strong impact on cell viability in the erlotinib condition (FIG. 4). At least three single siRNA had to have an effect based on the cut-offs define above in order for the gene to be scored as a positive hit in the screen.

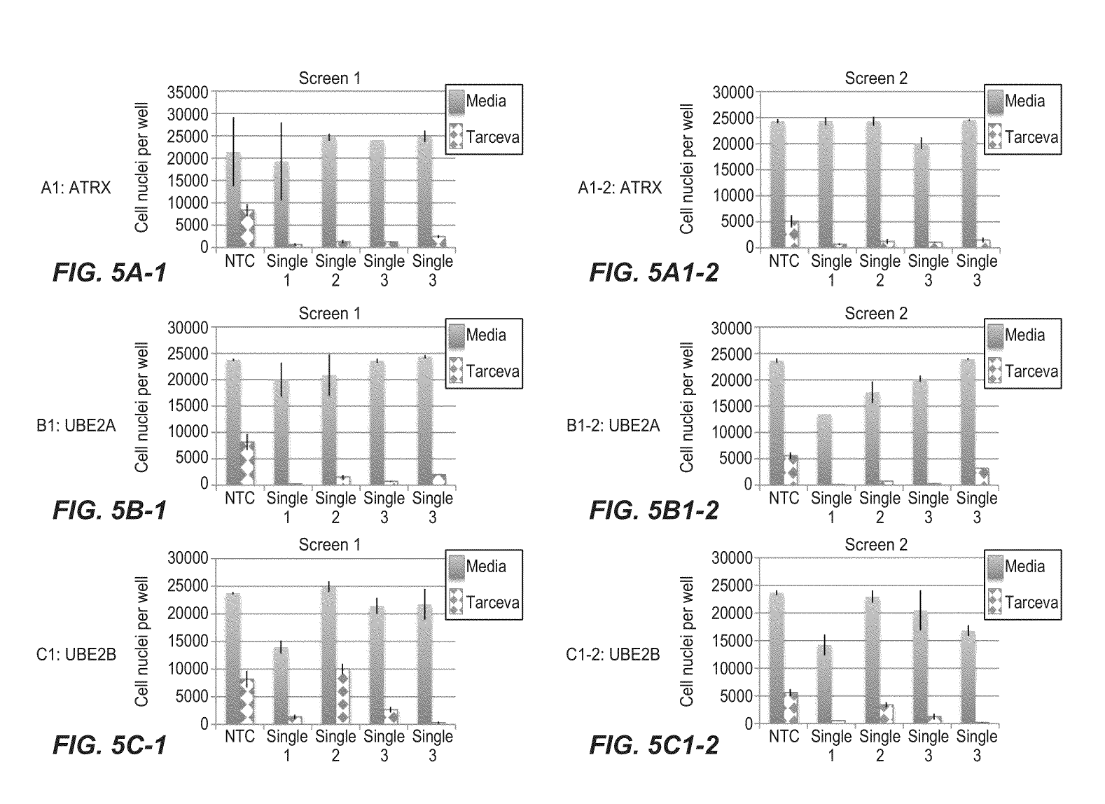

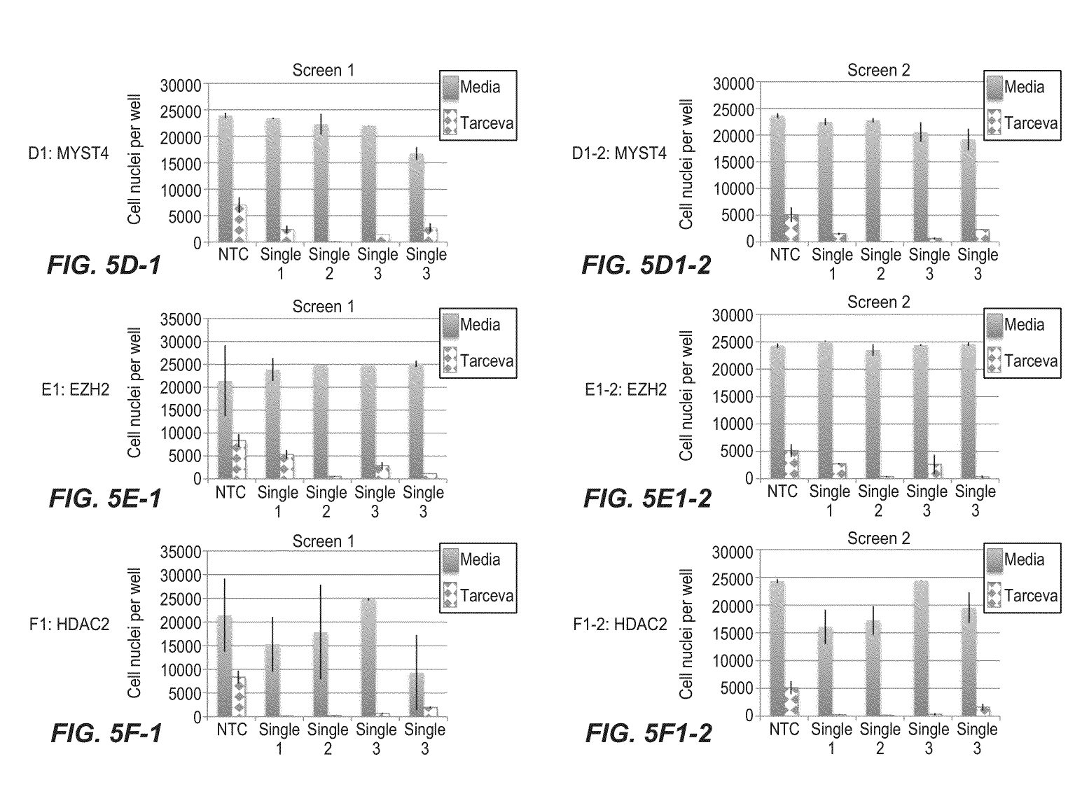

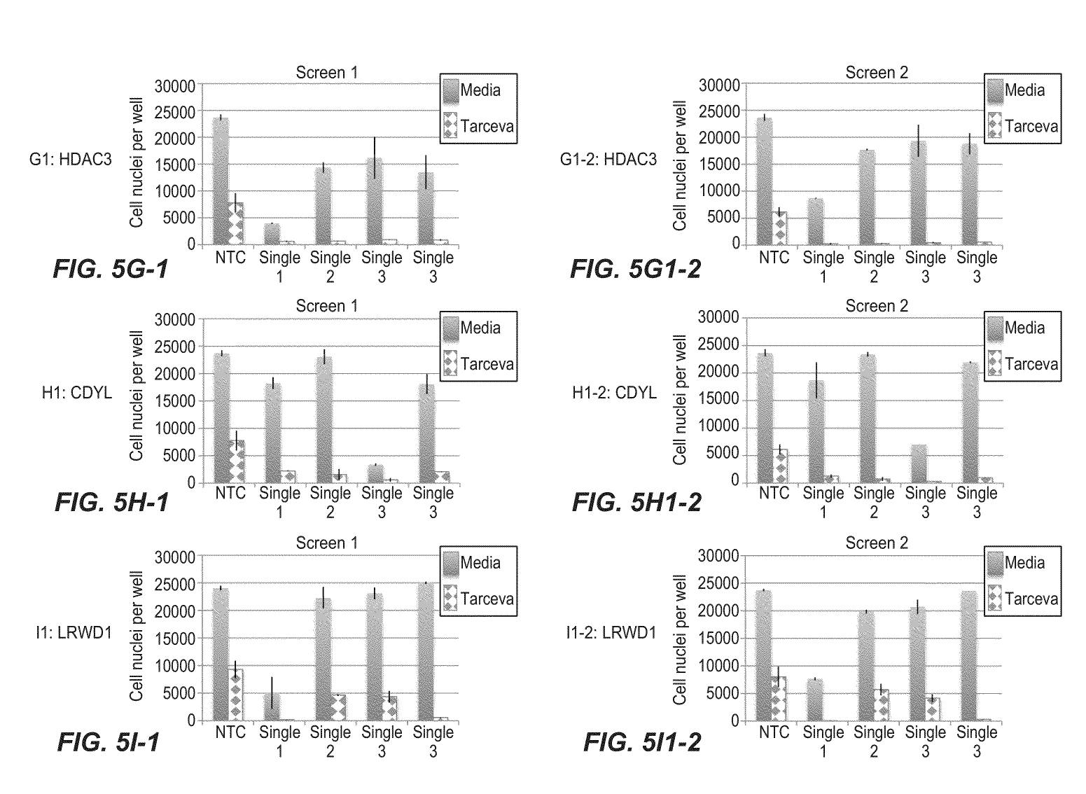

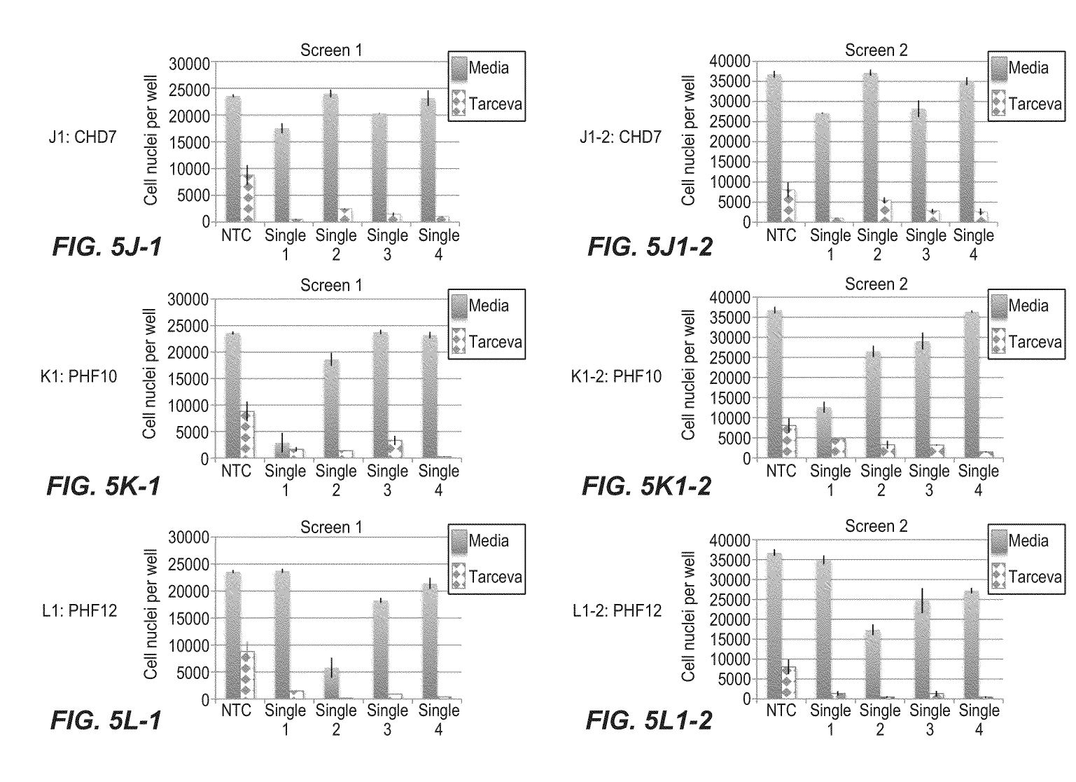

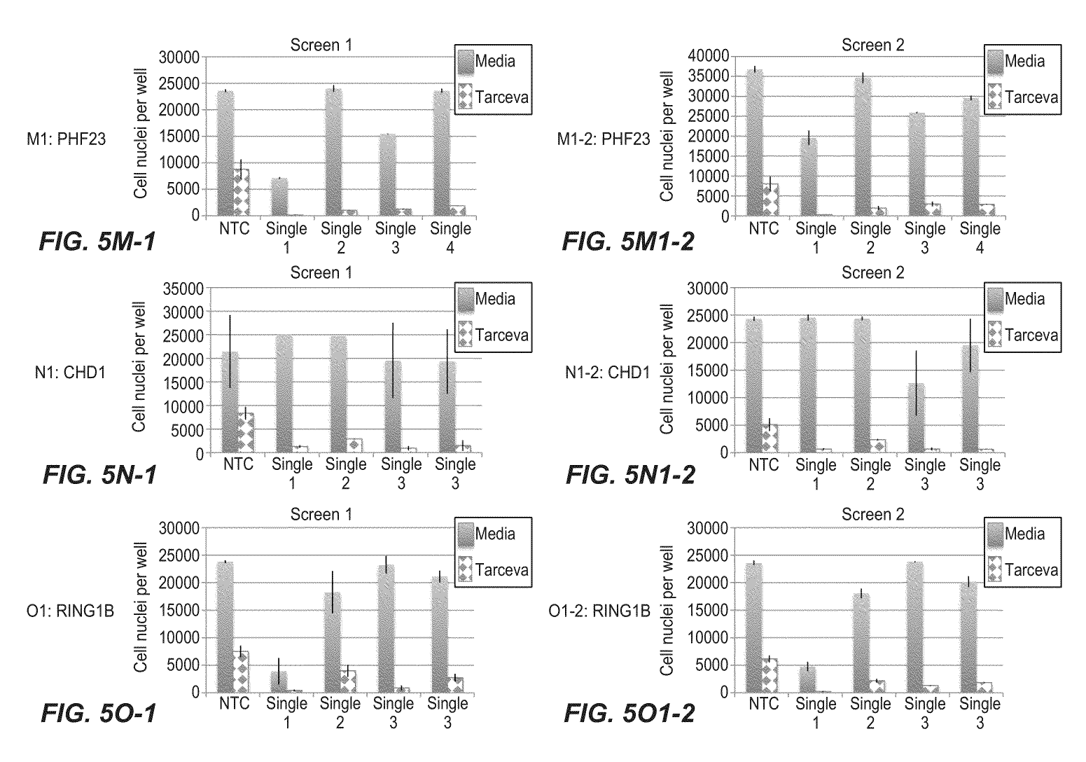

Targets were initially selected as positive siRNA hits. Raw data for each individual hit are described on FIG. 5A-1, FIG. 5A1-2, FIG. 5B-1, FIG. 5B1-2, FIG. 5C-1, FIG. 5C1-2, FIG. 5D-1, FIG. 5D1-2, FIG. 5E-1, FIG. 5E1-2, FIG. 5F-1, FIG. 5F1-2, FIG. 5G-1, FIG. 5G1-2, FIG. 5H-1, FIG. 5H1-2, FIG. 5I-1, FIG. 5I1-2, FIG. 5J-1, FIG. 5J1-2, FIG. 5K-1, FIG. 5K1-2, FIG. 5L-1, FIG. 5L1-2, FIG. 5M-1, FIG. 5M1-2, FIG. 5N-1, FIG. 5N1-2, FIG. 5O-1 and FIG. 5O1-2. None of the single siRNA targeting ATRX had any significant effect in the media condition while all of them significantly reduce cell viability in presence of erlotinib (FIG. 5A1-2). Similar 4 out of 4 positive siRNA results were observed for UBE2A (FIG. 5B1-2), MYST4 (FIG. 5D1-2), EZH2 (FIG. 5E1-2), CHD7 (FIG. 5J1-2), and CHD1 (FIG. 5N1-2). Other positive siRNA hits included UBE2B (FIG. 5C1-2), HDAC2 (FIG. 5F1-2), HDAC3 (FIG. 5G1-2), CDYL (FIG. 5H1-2), LRWD1 (FIG. 5I1-2), PHF10 (FIG. 5K1-2), PHF12 (FIG. 5L1-2), PHF23 (FIG. 5M1-2), and RING1B (FIG. 5O1-2) were classified as 3 out of 4 positive siRNA hits since one of the siRNA had some effect on cell viability in the media condition.

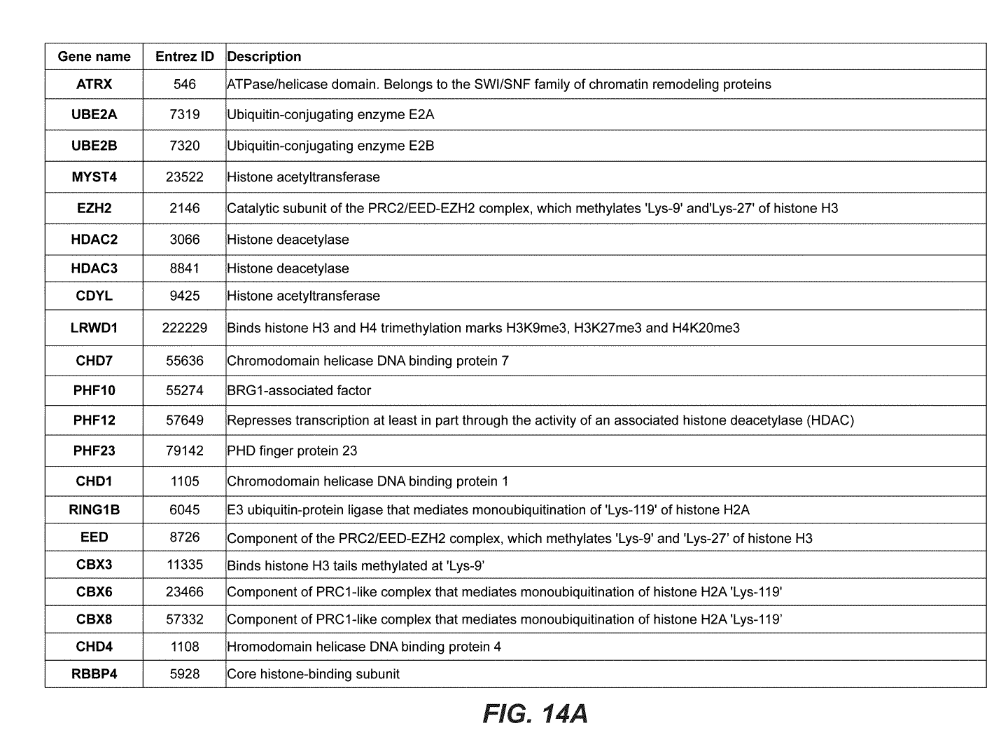

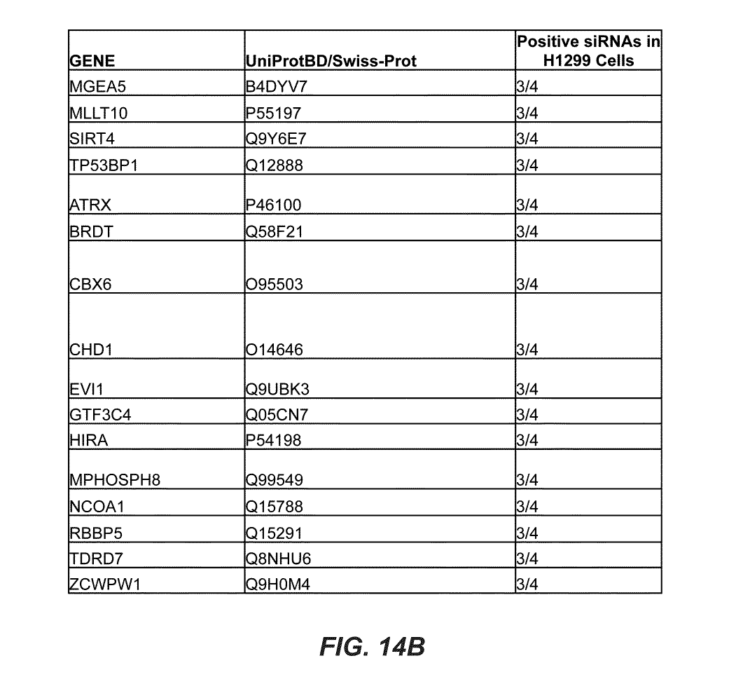

Based on the siRNA screen data the following genes were identified as being involved in the drug tolerant persister phenotype: ATRX, UBE2A, UBE2B, MYST4, EZH2, HDAC2, HDAC3, CDYL, LRWD1, CHD7, PHF10, PHF12, PHF23, CHD1, RING1B, EED, CBX3, CBX6, CBX8, CHD4, and RBBP4 as shown in FIG. 14A.

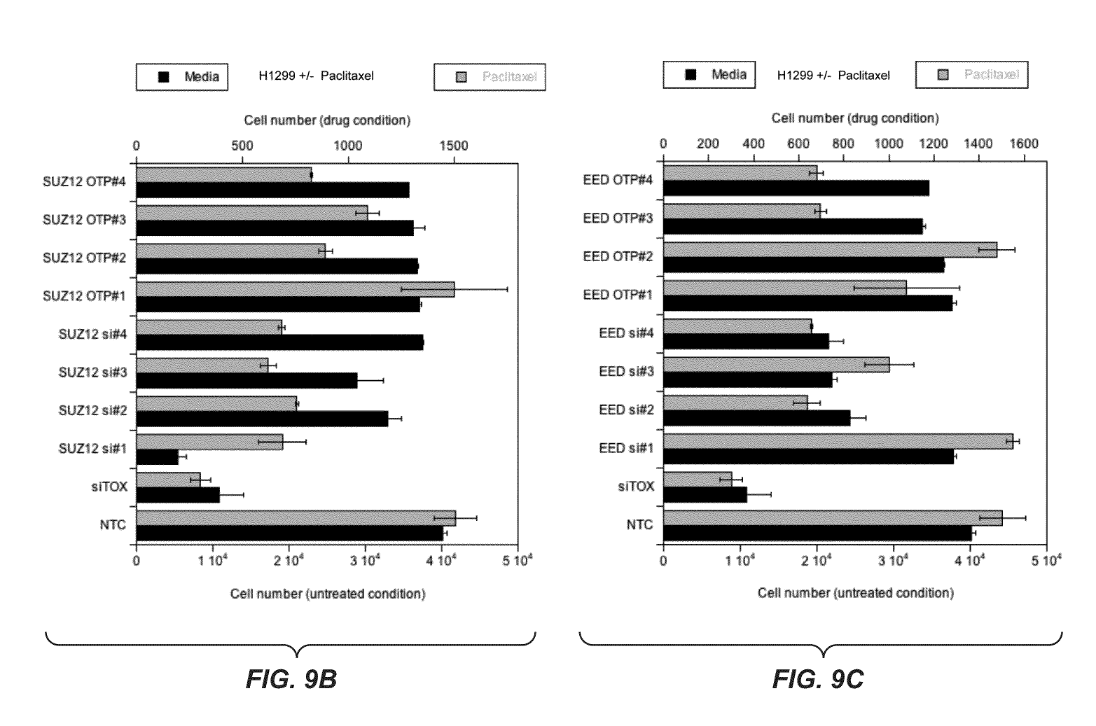

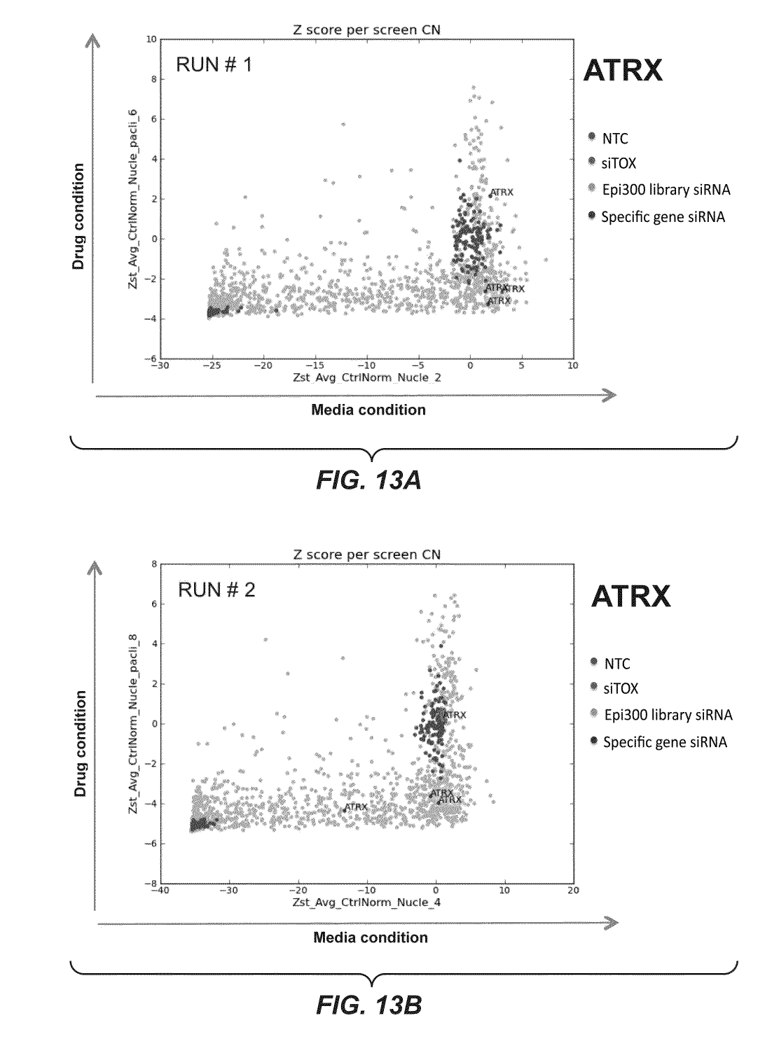

A second siRNA screen was developed and implemented using the human lung adenocarcinoma cancer cell line H1299 in the context of DTPs. H1299 DTP cells were prepared and screened as described above for the PC9/erlotinib screen using the taxane, paclitaxel, as the drug instead of erlotinib. Results for ATRX are shown in FIG. 13. As shown in FIG. 14B, the second siRNA screen in H1299 cells using the taxane, paclitaxel, identified genes involved in the drug tolerant persister phenotype: MGEA5, MLLT10, SIRT4, TP53BP1, ATRX, BRDT, CBX6, CHD1, EVI1, GTF3C4, HIRA, MPHOSPH8, NCOA1, RBBP5, TDRD7, and ZCWPW1. HDAC2 was positive 3 out of 4 siRNAs in the first run and 2 out of 4 in the second run tested in H1299 cells while HDAC3 was negative in H1299 treated with paclitaxel (data not shown). Further, in the H1299 cell line both EZH2 and SUZ12 were confirmed as hits (6 out of 8 positive siRNA hit) (FIG. 9).

### Polycomb Repressive Complex 1 and 2

Since RING1B and EZH2, components of the Polycomb Repressive Complex 1 (PRC1) and Polycomb Repressive Complex 2 (PRC2), respectively, were identified as positive hits in the siRNA screens in PC9 DTP cells further studies investigating the involvement of these complex components in drug tolerant persistence were performed.

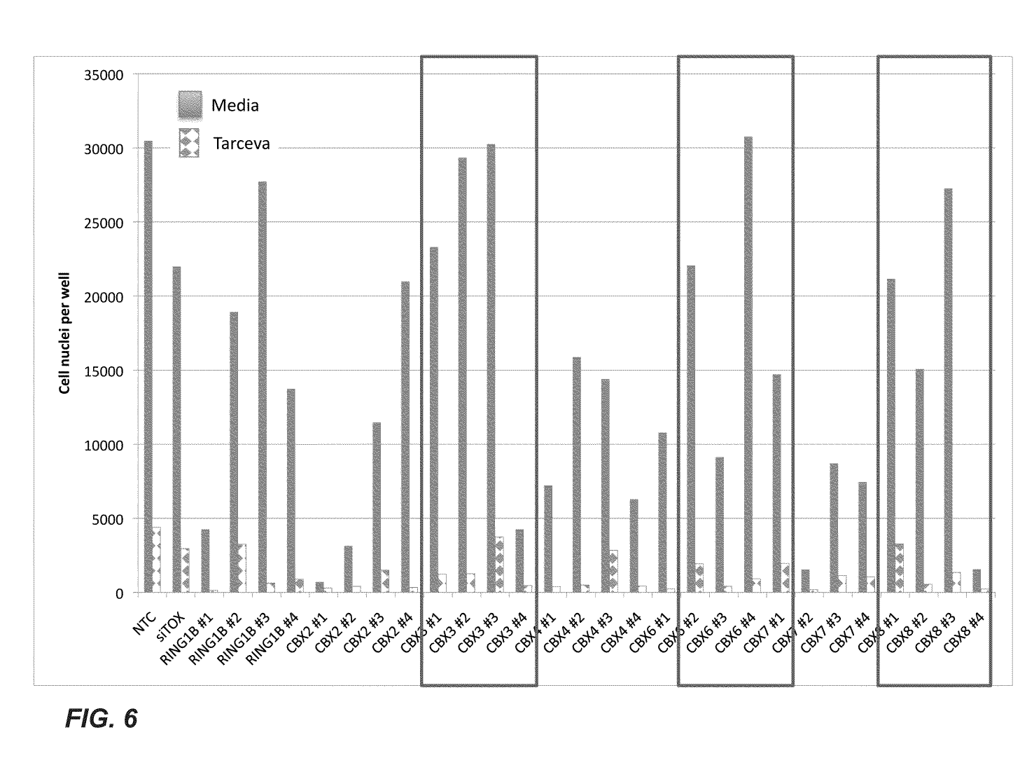

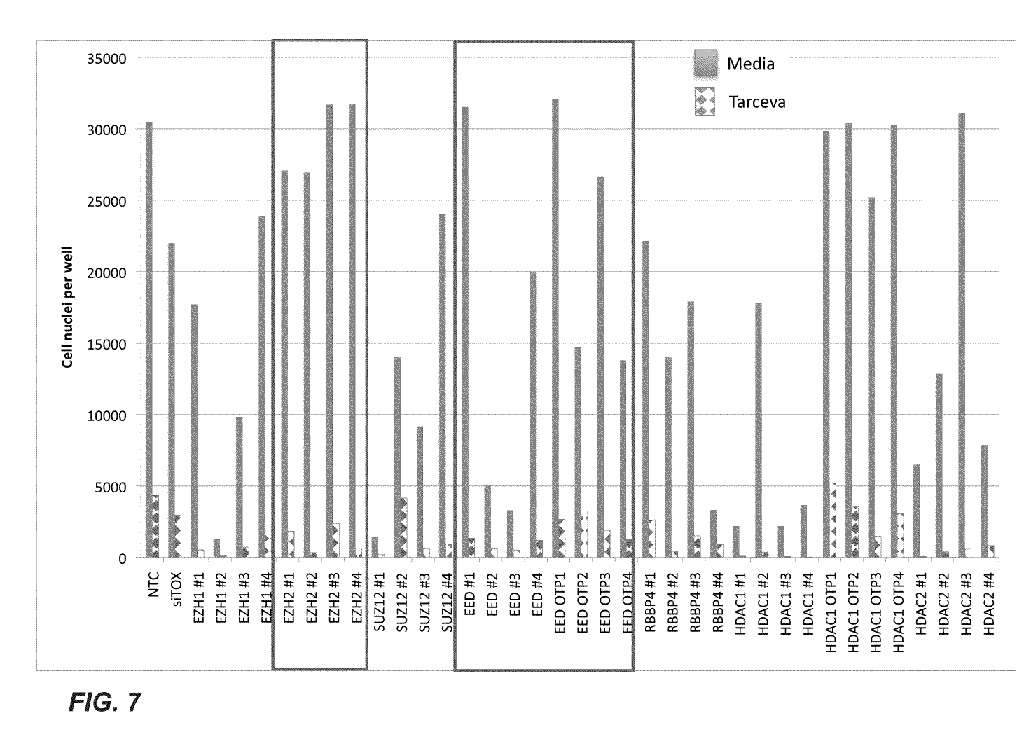

Several additional components of the PRC1 complex were knockdown down using 4 single siRNAs (Dharmacon siGENOME) per gene (FIG. 6). In addition to confirm RING1B as a 3 out of 4 positive siRNA hit, CBX3, CBX6 and CBX 8 also were implicated with at least 2 out of 4 siRNAs reducing cell viability in presence of erlotinib without any effect in media.

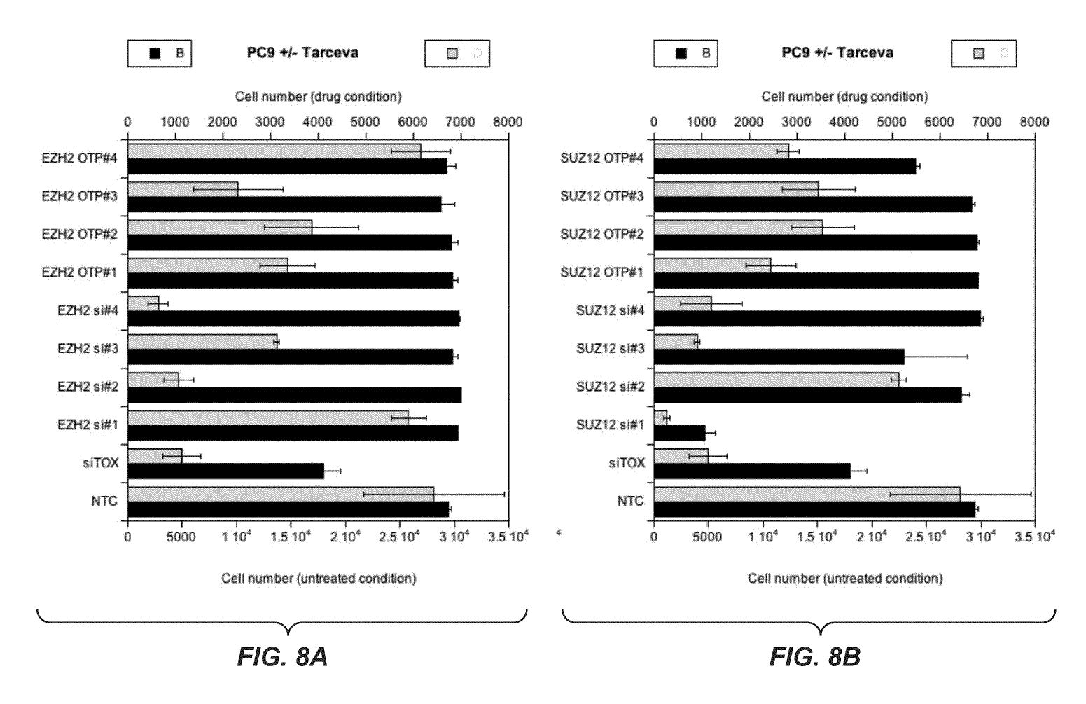

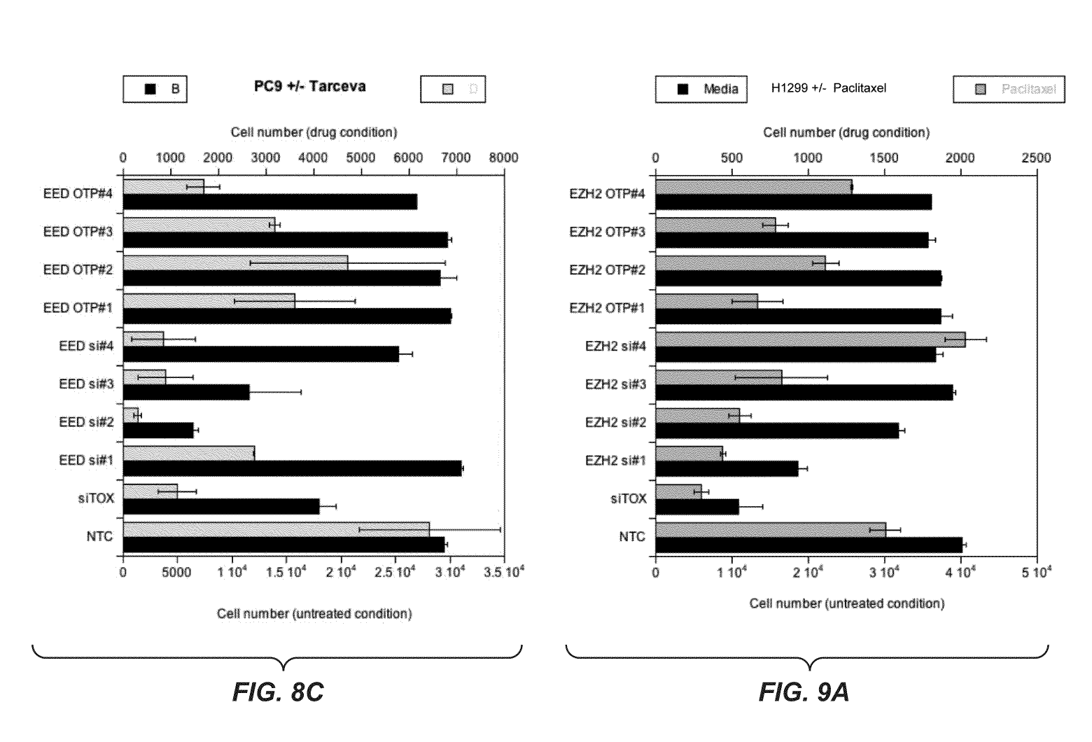

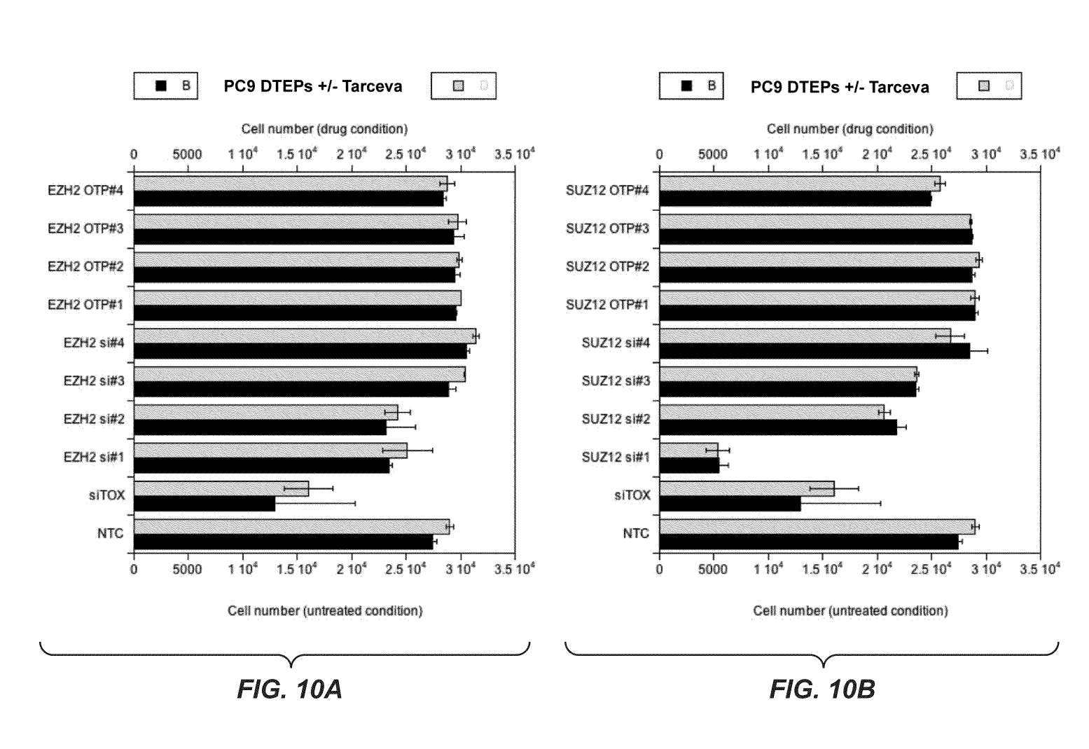

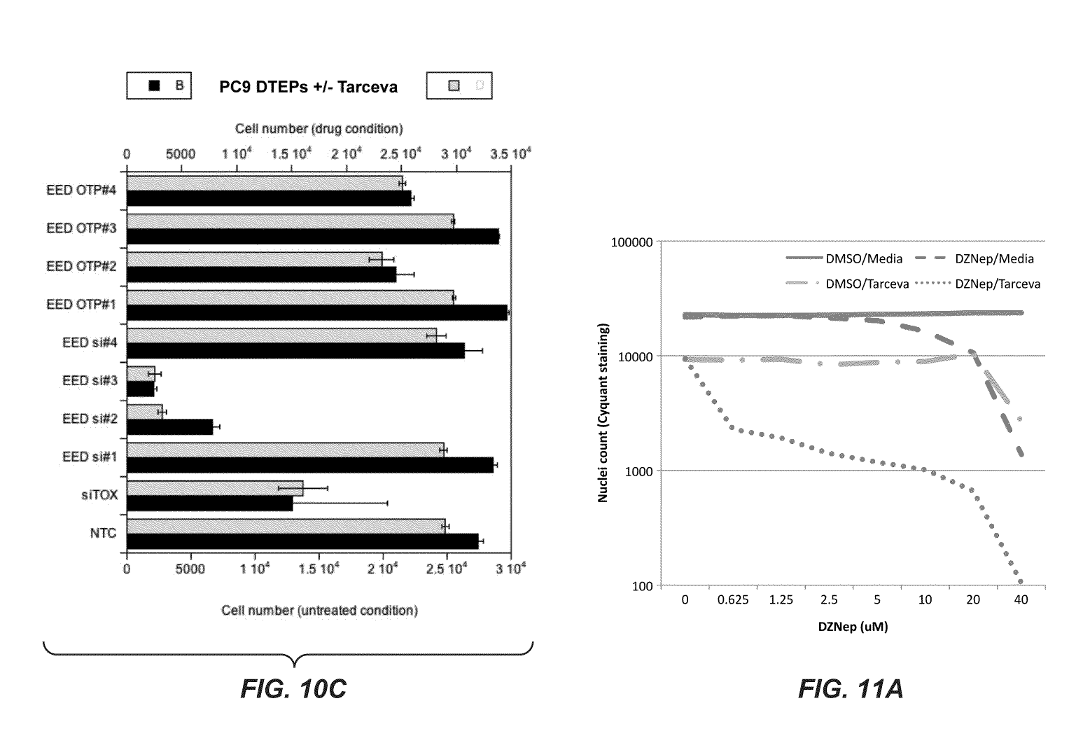

Several additional components of the PRC2 complex were also investigated. In addition to non-modified siRNA (Dharmacon SiGENOME), additional siRNA sequences were tested as modified siRNA (Dharmacon ON-TARGET PLUS) on PC9 cells for EZH2, EED and SUZ12 (FIG. 8). Using this approach EZH2 was confirmed as 6 out of 8, EED was a 5 out of 8 and SUZ12 was a 6 out of 8 positive siRNA hit in PC9 cells. The effect of knocking down the different PRC2 components in the human non-small cell lung carcinoma cell line H1299 treated with or without Paclitaxel (1 uM) (FIG. 9). In the H1299 cell line both EZH2 and SUZ12 were confirmed as hits (6 out of 8 positive siRNA hit). EED was not confirmed with only 2 out of 8 positive siRNA. Interestingly, none of these genes were positive hits in the PC9 DTEPs (FIG. 10).

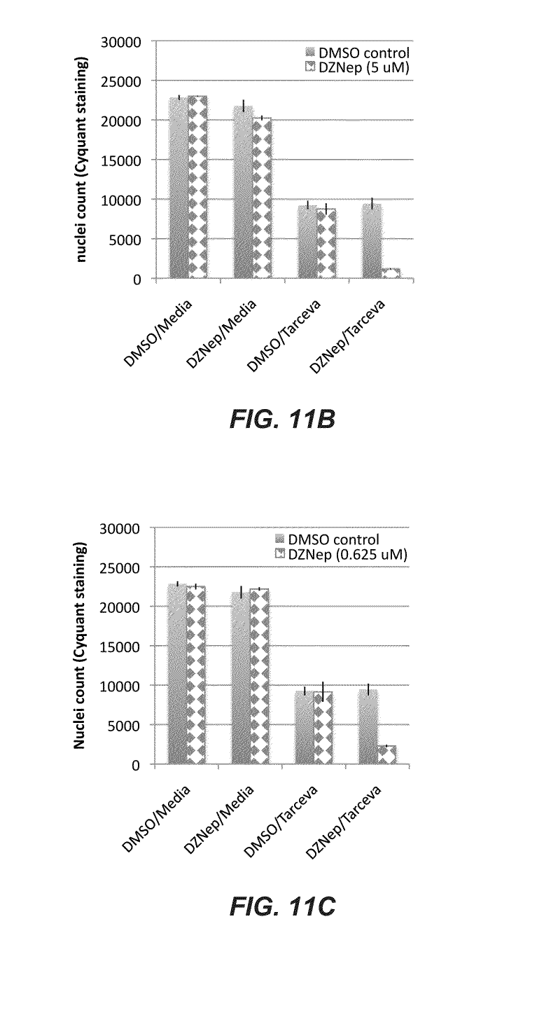

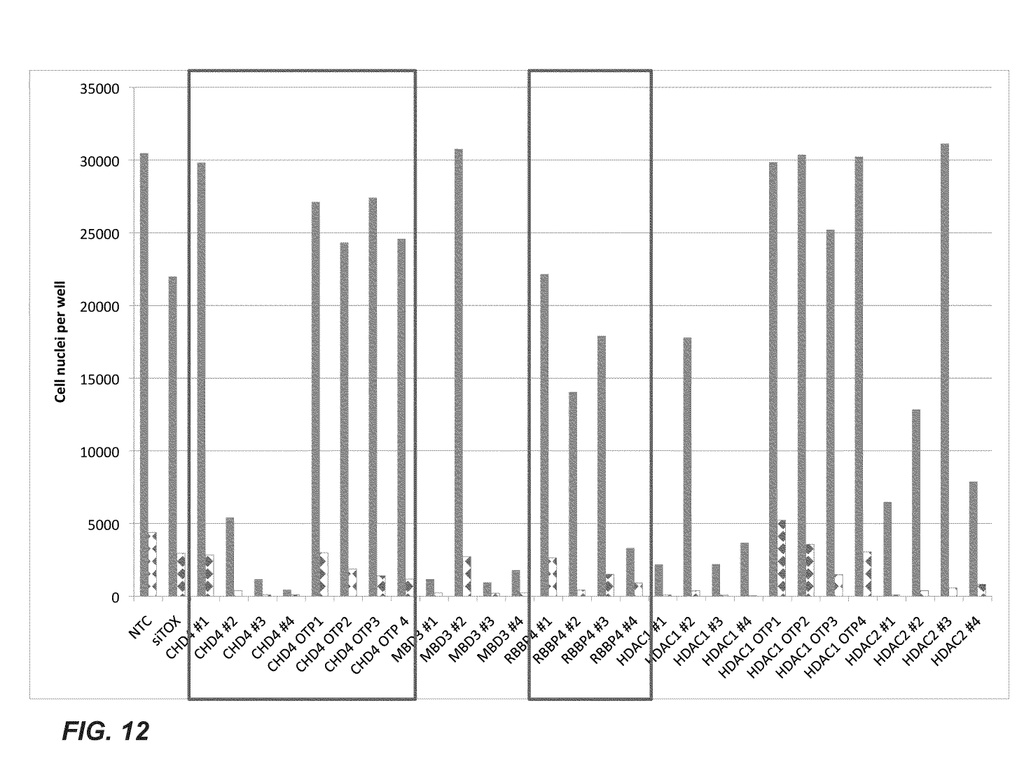

To further validate the involvement of PRC2 including EZH2 and EED in the maintenance of the DTP cells in response to erlotinib, the effect of the 3-deazaneplanocin A (DZNep), a histone methyltransferase inhibitor that has previously been shown to disrupt PRC2 by inhibiting EZH2, was tested as described above. As shown FIG. 11A, treatment of the PC9 DTP cells with increasing concentration of DZNep from 0.625 to 10 uM had a minimum impact on the cell viability in media alone. Significant effect of DZNep as a single agent was observed for concentrations equal to 20 and 40 uM on PC9 DTP cells. In combination with erlotinib, DZNep very significantly decrease the PC9 DTP cell viability with concentrations as low as 0.625 uM. Although DZNep has not effect by itself on PC9 DTP cell viability at 0.625 (FIG. 11B) and 5 uM (FIG. 11C), these concentrations results in 75.8 and 87.5% cell killing, respectively, when compared to erlotinib alone. These results correlated with the effect of EZH2 and EED knockdown, and further establish the implication of PRC2 in the maintenance of the DTPs.

### Nucleosome Remodeling and Histone Deacetylase NuRD Complex

Several components of the NuRD complex were also investigated using multiple single siRNAs (Dharmacon siGENOME or ON-TARGET PLUS). Among the different components investigated 4 out of 8 single siRNAs targeting CHD4 clearly decreased the PC9 DTP cell viability in presence of erlotinib while having no effect in media. This demonstrated the involvement of CHD4 in the maintenance of the DTPs. Three out of 4 single siRNAs targeting RBBP4 had a slight effect on PC9 DTP cell viability in media while significantly decreasing PC9 DTP cell survival in presence of erlotinib. This strongly suggests the implication of RBBP4 in the maintenance of the DTPs as well.

### Histone Mass Spectrometry Sample Preparation and Analysis

PC9 and PC9 DTP cell samples were prepared and analyzed as described as above. The studies showed significant alterations in histone tail modifications. Specifically, there was a change in acetylation pattern of histone H3 at lysine resides K9, K18, and K27. Further, there was in change in methylation pattern of histone H3 at lysine residues K4, K9, and K27.

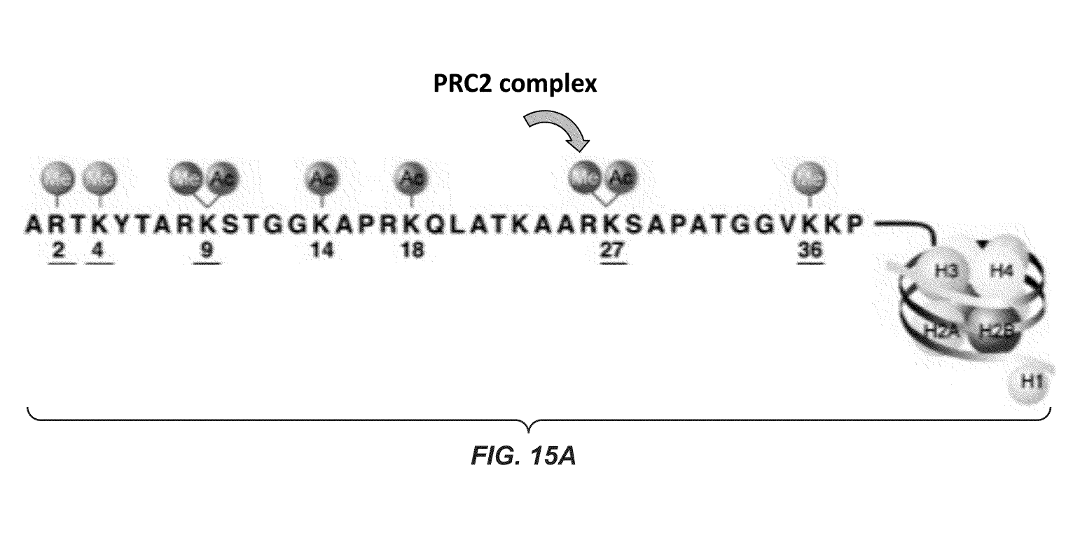

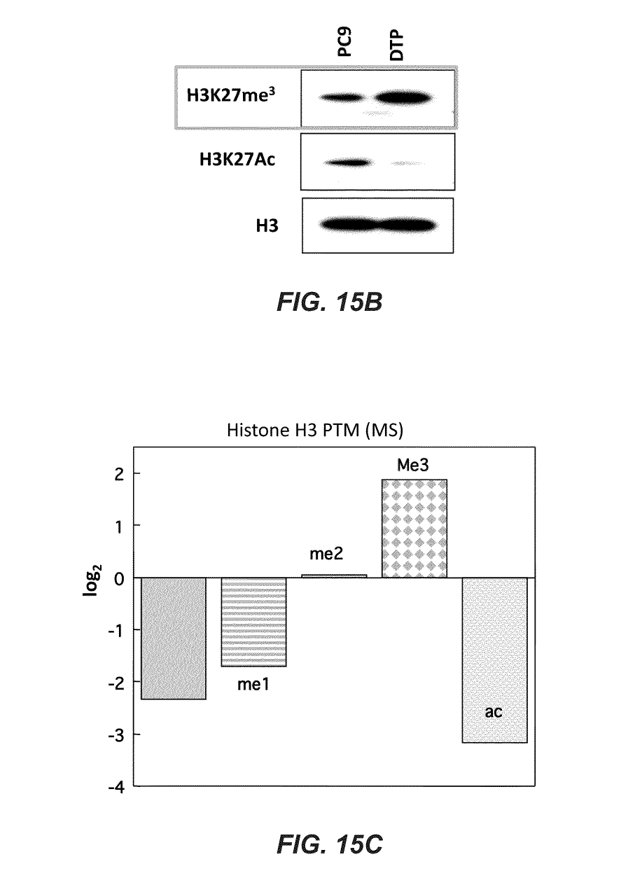

The histone post-translational modifications identified by histone mass spectrometry were consistent with the positive siRNA screen hits. In particular, the positive siRNA screen hits were modulators of specific histone H3 modifications altered in PC9 DTP cells compared to PC9 as determined by mass spectrometry. For example, histone H3K4 methylation was lowered (e.g., increase in unmethylated, mono and di-methylated as compared to tri-methylated) and histone H3K9 methylation was increased (e.g., increase in tri-methylated as compared to di, mono or un-methylated) in PC9 DTP cells compared to PC9 cells as determined by mass spectrometry. Consistent with this finding, the positive siRNA screen hit, ATRX, is a reader of low histone H3K4 methylation and high histone H3K9 methylation, and CHD7, another positive siRNA screen hit, is a potential reader of histone H3K4 un-methylated. Similarly, the positive siRNA screen PRC1 component hits, Ring1B and CBX proteins, read methylated histone H3K27 which was increased (e.g., increase in tri-methylated as compared to di, mono, or un-methylated) in PC9 DTP cells compared to PC9 cells as shown by Western blot and mass spectrometry. See FIG. 15B and FIG. 15C. Further and consistent with the change in methylated histone H3K27, histone H3K27 acetylation pattern was reduced as shown by Western blot and mass spectrometry. See FIG. 15B and FIG. 15C. Reduction of H3K4 trimethylation and increase in H3K9 trimethylation and H3K27 trimethylation was confirmed by mass spectrometry and Western blot (data herein and data not shown).

### Role of Histone Deacetylases in Drug Tolerance

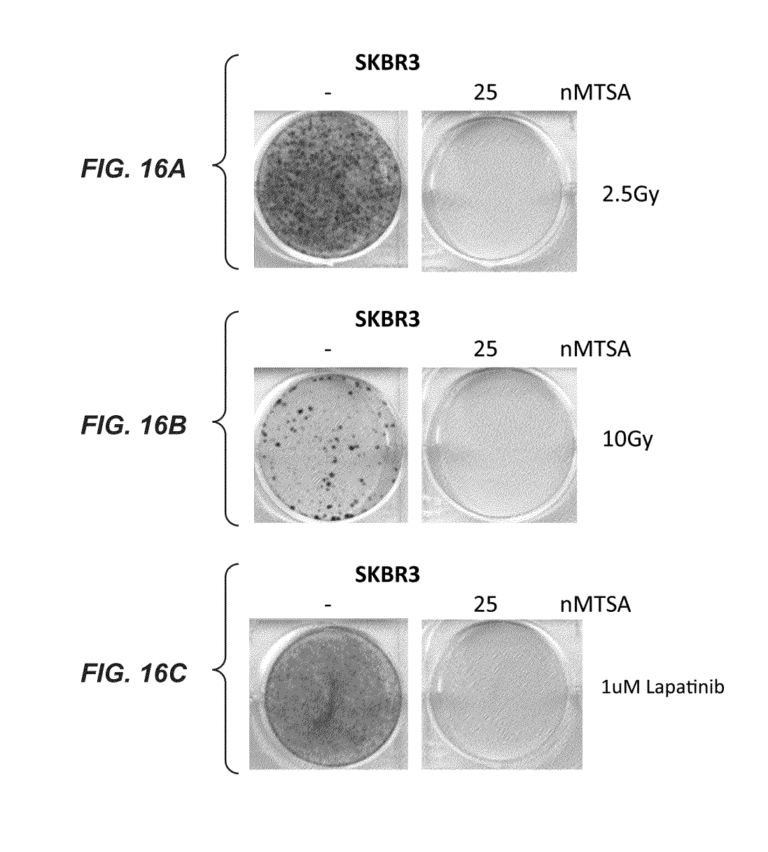

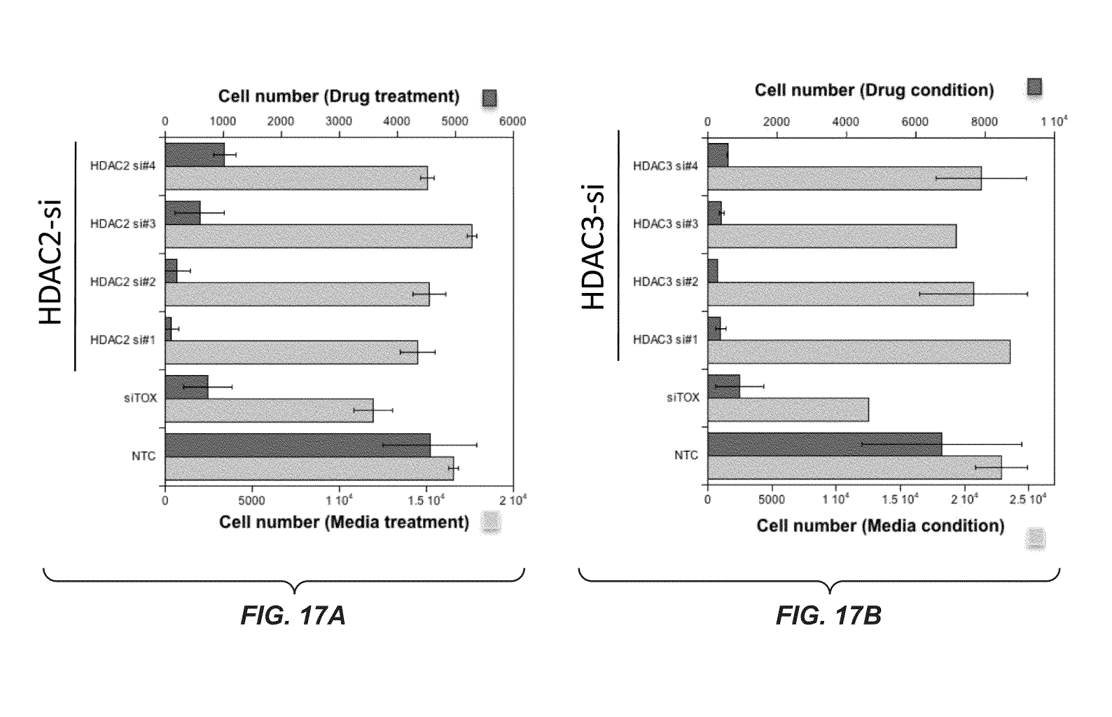

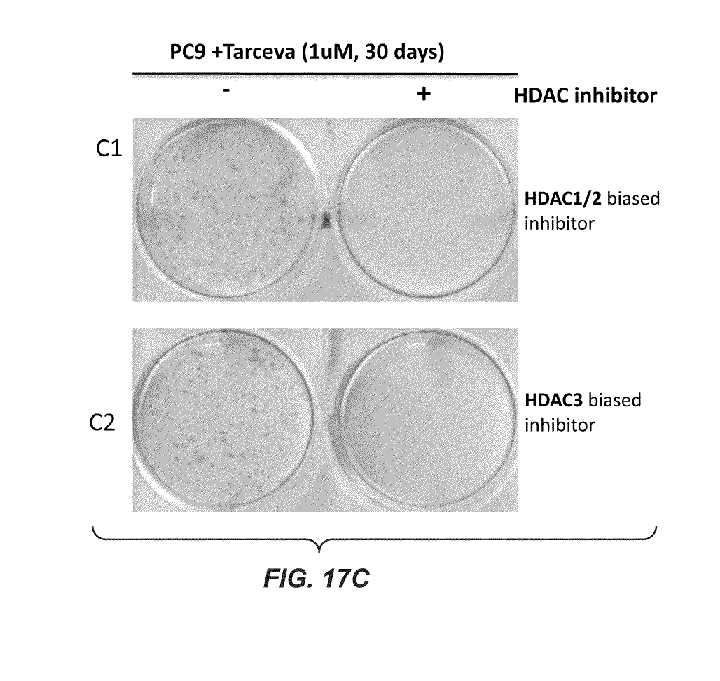

The role of histone deacetylases was further test in additional models to elucidate their role in drug tolerance. The class I and II HDAC inhibitor TSA was tested in SKBR3 cells in combination with radiotherapy at 2.5 Gy and 10 Gy. As shown in FIG. 16A and FIG. 16B, the HDAC inhibitor TSA had a significant effect and eliminated radiotherapy drug tolerant cells. Similarly, TSA was shown in FIG. 17C to have a significant effect on lapatinib sensitivity and DTP formation.

To further investigate the role of HDACs in the establishment of drug tolerance, siRNA against HDAC2 and 3 as well as inhibitors that are HDAC1/2 or 3 biased were tested for their ability to disrupt the drug-tolerant state. As shown in FIG. 17A and FIG. 17B, siRNA knockdown of HDAC2 and HDAC3 expression resulted in a significant decrease in PC9 DTP formation in combination with erlotinib. In addition, as shown in FIG. 17C, HDAC small molecule inhibitors G946, HDAC1/2 biased inhibitor, and G877, HDAC3 biased inhibitor, were effective in reducing cell growth of PC9 DTP with erlotinib.

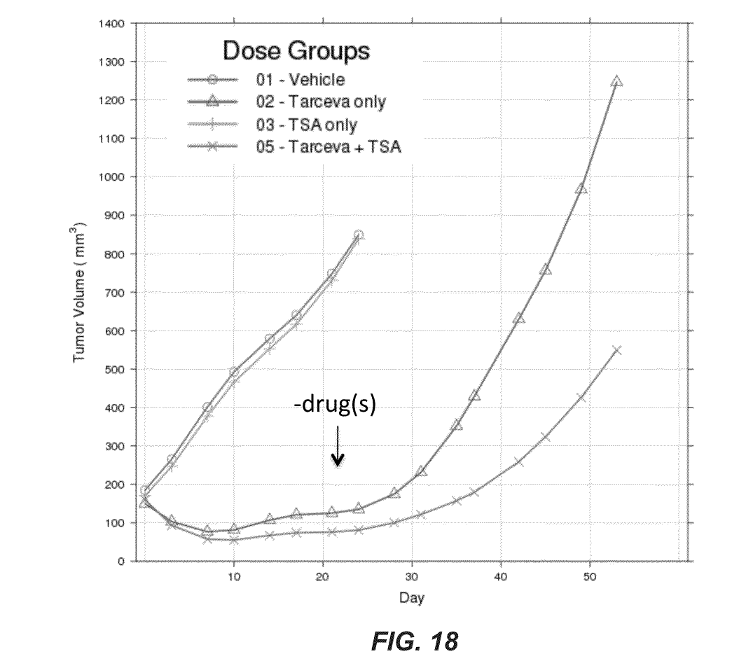

For the PC9 xenograft study the mice were inoculated with PC9 cells and the tumors were allowed to grow to 100-200 mm³ in size, which were then divided into four treatment groups, namely, vehicle control, trichostatin A (TSA) control, erlotinib alone, and erlotinib+TSA groups. While TSA alone had no effect on tumor growth, the combination of erlotinib+TSA resulted in a substantial delay in tumor relapse as shown in FIG. 18.

### Role of PRC2 and EZH2 in Drug Tolerance

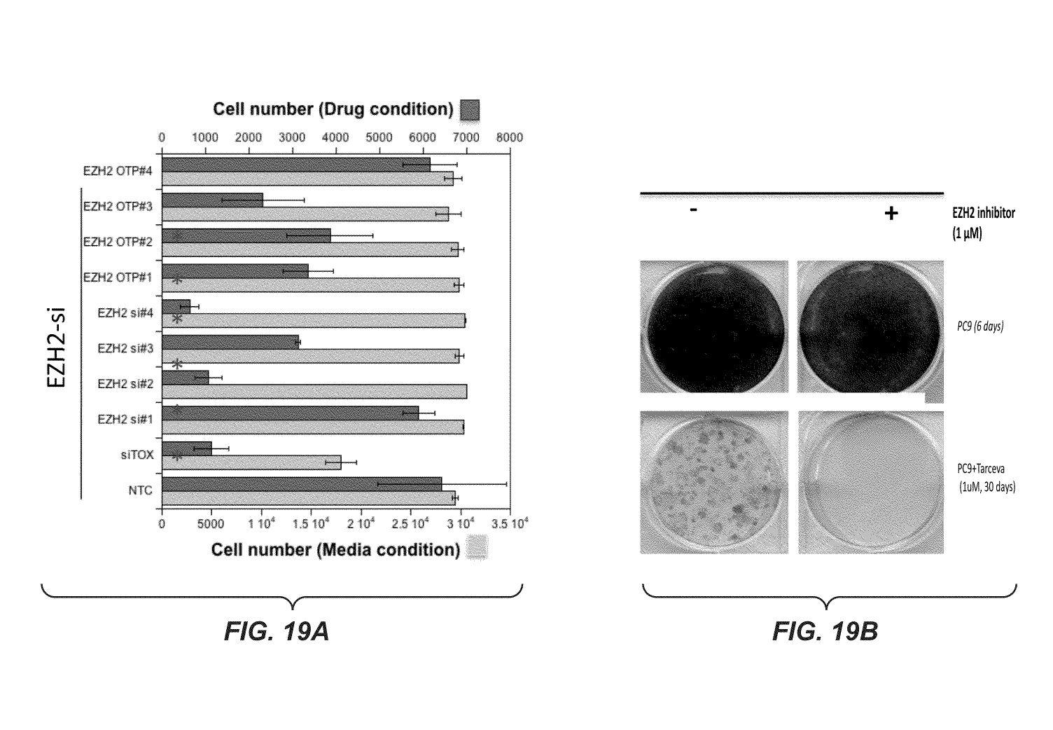

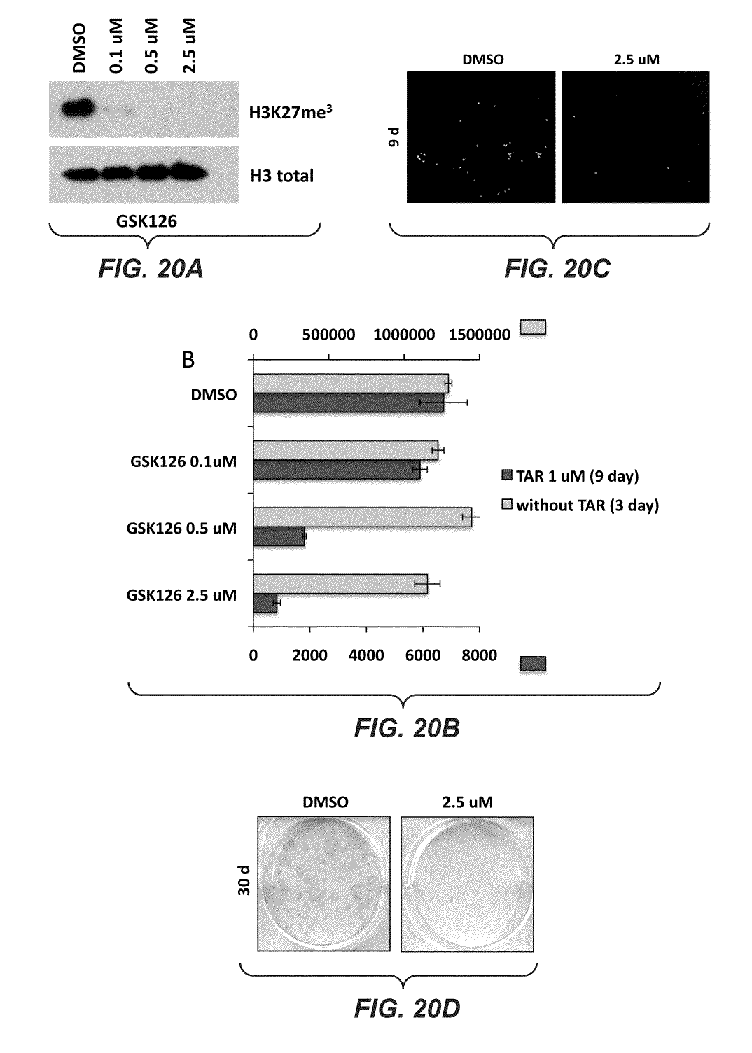

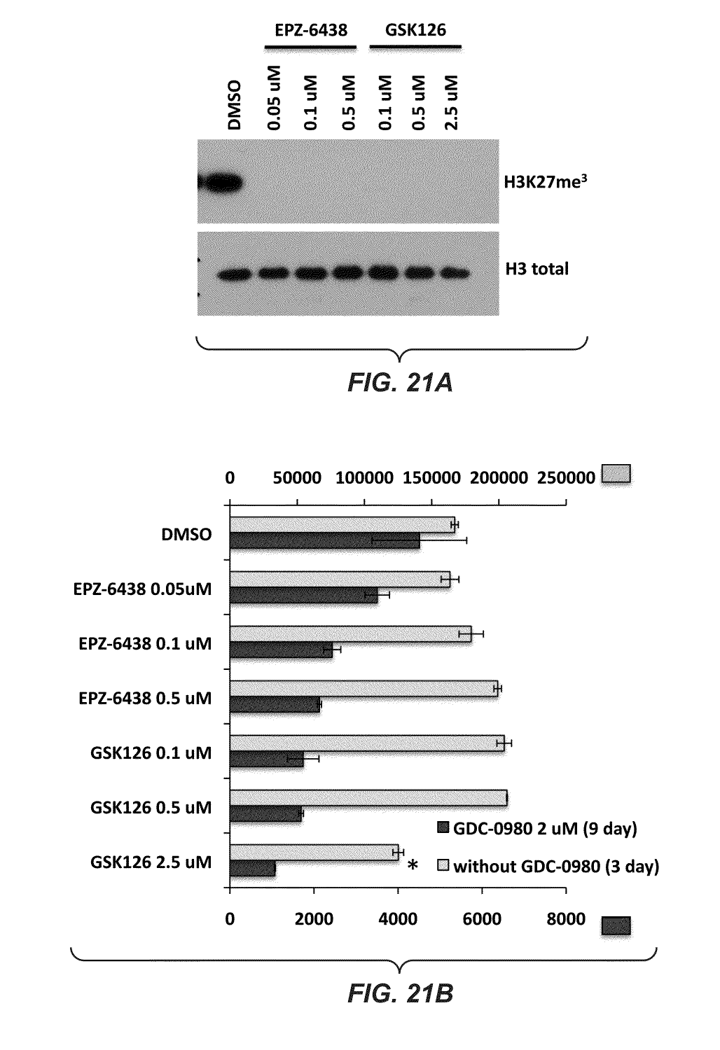

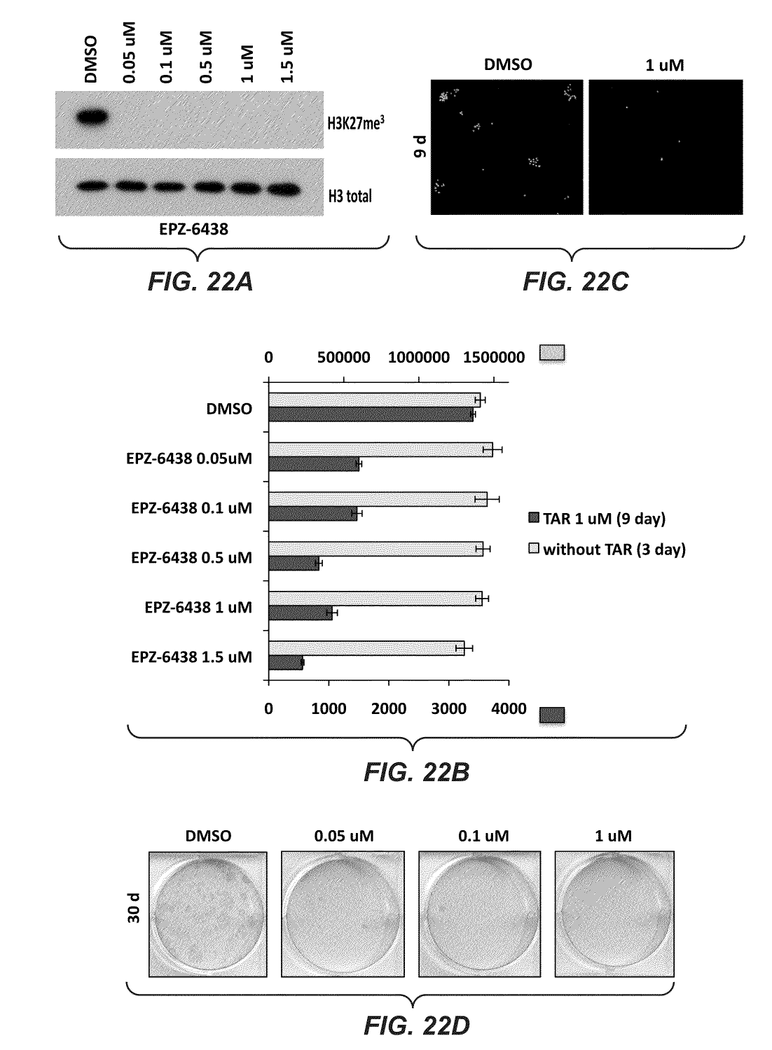

To further investigate the role of EZH2 in the establishment of drug tolerance, siRNA against EZH2 as well as a small molecule inhibitor were tested for their ability to disrupt the drug-tolerant state. As shown in FIG. 19A, siRNA knockdown of EZH2 expression resulted in a significant decrease in PC9 DTP formation in combination with erlotinib. GSK126 and EPZ-6438, as shown in FIG. 20A, FIG. 21A, and FIG. 22A are effective in reducing H3K27 trimethylation in PC9 cell treated with Tarceva and in EVSAT cells treated with the PI3 kinase inhibitor, GDC-0908. In addition, as shown in FIG. 19B, FIG. 20B, FIG. 20C, and FIG. 20D, EZH2 small molecule inhibitor GSK126 was effective in reducing cell growth in a dose dependent manner of PC9 DTP with erlotinib. Similarly, as shown in FIG. 22B, FIG. 22C, and FIG. 22D, the EZH2 small molecule inhibitor EPZ-6438 was effective in reducing cell growth in a dose dependent manner of PC9 DTP with erlotinib. The EZH2 inhibitors were effective on other drug tolerance models such as breast cancer cell line EVSAT (red) GDC-0908 DTP, breast cancer cell line SKBR3 (red) lapatinib DTP, breast cancer cell line BT474 (red) lapatinib DTP, the melanoma cell line M14 Mek inhibitor/paclitaxel DTP, and colon cancer cell line colo205 AZ628 DTP. For example as shown in FIG. 21B, EZH2 small molecule inhibitors, GSK126 and EPZ-6438 were effective in reducing cell growth in a dose dependent manner of EVSAT DTP with the PI3 kinase inhibitor GDC-0980.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1) A method of treating cancer in an individual comprising administering to the individual (a) a modulator of a chromatin modifier and (b) an EGFR antagonist or a taxane.

2) The method of claim 1, wherein the respective amounts of the modulator of the chromatin modifier and the EGFR antagonist or taxane are effective to increase the period of cancer sensitivity and/or delay the development of cell resistance to the EGFR antagonist or taxane.

3) The method of claim 1, wherein the modulator of the chromatin modifier is an antagonist of the chromatin modifier.

4) The method of claim 1, wherein the modulator of the chromatin modifier is an antibody inhibitor, a small molecule inhibitor, a binding polypeptide inhibitor, and/or a polynucleotide antagonist.

5) The method of claim 1, wherein the modulator of the chromatin modifier) is an antagonist of a member of polycomb repressive complex 2 (PRC2).

6) The method of claim 5, wherein the antagonist of a member of PRC2 is an antagonist of EZH2, EED, and/or SUZ12.

7) The method of claim 1, wherein the modulator of the chromatin modifier is an antagonist of a member of polycomb repressive complex 1 (PRC1).

8) The method of claim 7, wherein the antagonist of a member of PRC1 is an antagonist of RING1B, CBX3, CBX6, and/or CBX8.

9) The method of claim 1, wherein the modulator of the chromatin modifier is an antagonist of a member of NURD complex.

10) The method of claim 9, wherein the antagonist of a member of NURD complex is an antagonist of CHD4 and/or RBBP4.

11) The method of claim 1, wherein the modulator of the chromatin modifier (e.g., antagonist of the chromatin modifier) is an antagonist of HDAC1, HDAC2, and/or HDAC3.

12) The method of claim 1, wherein the modulator of the chromatin modifier is an antagonist of one or more of ATRX, MYST4, CDYL, LRWD1, CHD7, PHF10, PHF12, PHF23, CHD1, MGEA5, MLLT10, SIRT4, TP53BP1, ATRX, BRDT, CBX6, CHD1, EVI1, GTF3C4, HIRA, MPHOSPH8, NCOA1, RBBP5, TDRD7, and ZCWPW1.

13) The method of claim 1, wherein the method comprises EGFR antagonist.

14) The method of claim 13, wherein the EGFR antagonist is N-(3-ethynylphenyl)-6,7-bis(2-methoxyethoxy)quinazolin-4-amine or a pharmaceutically acceptable salt thereof.

15) The method of claim 13, wherein the EGFR antagonist is gefitinib and/or erlotinib

16) The method of claim 1, wherein the method comprises taxane.

17) The method of claim 16, wherein the taxane is paclitaxel or docetaxel.

18) The method of claim 1, wherein the modulator of the chromatin modifier (e.g., antagonist of the chromatin modifier) and the EGFR antagonist or taxane is administered concomitantly.

19) The method of claim 1, wherein the cancer is lung cancer (e.g., non-small cell lung cancer (NSCLC) and/or breast cancer.
